(19) United States
(12) Patent Application Publication
(10) Pub. No.: US 2023/0116240 A1
(43) Pub. Date: Apr. 13, 2023

(54) IMPROVEMENTS RELATING TO GAS MONITORING

(71) Applicant: Fisher & Paykel Healthcare Limited, Auckland (NZ)

(72) Inventors: Matthew Jon PAYTON, Auckland (NZ); Thomas Heinrich BARNES, Surrey (GB)

(21) Appl. No.: 17/906,082

(22) PCT Filed: Mar. 12, 2021
(86) PCT No.: PCT/IB2021/052062

§ 371 Date: Sep. 12, 2022

Related U.S. Application Data
(60) Provisional application No. 62/989,081, filed on Mar. 13, 2020.

(51) Int. Cl. A61M 16/00; A61M 16/12

(52) U.S. Cl. CPC A61M 16/024; A61M 16/12; A61M 2205/502; A61M 2230/43; A61M 2230/42

(57) ABSTRACT

Disclosed herein is a method and apparatus for determining a parameter of a gas present in an exhaled gas flow comprising: providing an apparatus gas flow with a time-varying parameter to a patient, measuring a parameter of the gas present in a composite gas outflow from the patient, and determining the parameter of the gas present in the exhaled gas flow using the measured parameter of the gas present in the composite gas outflow and the time-varying parameter.

### FIELD OF THE INVENTION

The present invention relates to a method and apparatus for determining a parameter of a gas flow exhaled by a patient when using a respiratory apparatus.

### BACKGROUND OF THE INVENTION

When providing flow support/therapy to a patient, clinicians often monitor patient exhaled gas parameters such as O₂ fraction and/or CO₂ fraction. Due to mixing of various flows, the monitored gas parameters are often not a true reflection of the actual exhaled gas parameters.

### SUMMARY OF INVENTION

It is an object of the present invention to provide an apparatus and/or method to obtain an estimate of a parameter of a gas exhaled by a patient.

In one aspect the present invention may be said to comprise a method of determining a parameter of a gas present in an exhaled gas flow comprising: providing an apparatus gas flow with a time-varying parameter to a patient, measuring a parameter of the gas present in a composite gas outflow from the patient, and determining the parameter of the gas present in the exhaled gas flow using the measured parameter of the gas present in the composite gas outflow and the time-varying parameter.

In another aspect the present invention may be said to comprise an apparatus for providing an apparatus gas flow and determining a parameter of an exhaled patient gas flow, comprising: a flow source, a sensor for sensing a composite gas outflow, and a controller, wherein the apparatus is configured to: provide an apparatus gas flow with a time-varying parameter, determine a parameter of a gas present in a composite gas outflow from the patient, the composite gas outflow comprising: leak gas flow from apparatus gas flow, and exhaled gas flow from a patient with the gas, And determine the parameter of the gas present in the exhaled gas flow using the determined gas parameter and the time-varying parameter.

Optionally the time varying parameter is one or more of: flow rate of the apparatus gas flow, or gas proportion, where optionally the gas proportion is: a fraction of a gas present in the apparatus gas flow, a partial pressure of a gas present in the apparatus gas flow.

Optionally the gas proportion is: gas fraction, preferably O₂ fraction; or gas partial pressure, preferably O₂ partial pressure.

Optionally the method or apparatus comprises providing the apparatus gas flow during an anaesthetic procedure.

Optionally the apparatus gas flow is provided via a non-sealing patient interface, preferably a non-sealing cannula.

Optionally the apparatus gas flow is a high flow gas flow.

Optionally the method or apparatus further comprises humidifying the apparatus gas flow.

Optionally determining the parameter of the gas present in the exhaled gas flow using the measured parameter of the gas present in the composite gas outflow comprises measuring just one gas and just the time varying parameter, the time varying parameter being flow rate.

In another aspect the present invention may be said to comprise a method of determining a parameter of a gas present in an exhaled gas flow comprising: providing an apparatus gas flow with a time-varying flow rate to a patient, determining a parameter of the gas present in a composite gas outflow from the patient, the patient composite gas outflow comprising: leak gas flow from apparatus gas flow, and exhaled gas flow from a patient with the gas, and determining the parameter of the gas present in the exhaled gas flow using the determined parameter of the gas present in the composite gas outflow and the time-varying flow rate.

Optionally the apparatus gas flow with the time-varying flow rate comprises at least a first flow rate at a first time and a second flow rate at a second time, and determining the parameter of the gas present in the exhaled gas flow using the determined parameter of the gas present in the composite gas outflow and the time-varying flow rate comprises: using the determined parameter of the gas present in the composite outflow determined at the first flow rate and determined at the second flow rate.

Optionally the parameter comprises a fraction of the gas component in the exhaled gas flow.

Optionally the gas is: CO₂, O₂, Nitrogen, Helium and/or Anaesthetic agent such as sevoflurane, and/or the sensor is configured to sense one or more of the following in the composite gas outflow: CO₂, O₂, Nitrogen, Helium, and/or anaesthetic agent such as sevoflurane.

Optionally the parameter of the gas present in the composite gas outflow is determined during an inspiration and/or expiration phase of the patient breathing.

Optionally the parameter of gas present in an exhaled gas flow is gas fraction and determining the fraction of the gas (F_{E}) present in an exhaled gas flow using the determined parameter of gas present in the composite gas outflow and the time-varying flow rate comprises using:

$${F}_{E} ( t ) = \frac{{Q}_{o} ( t ) {F}_{m} ( t + Δ t ) ( {F}_{o} ( t ) - {F}_{m} ( t ) ) - {Q}_{o} ( t + Δ t ) {F}_{m} ( t ) ( {F}_{o} ( t + Δ t ) - {F}_{m} ( t + Δ t ) )}{{Q}_{o} ( t ) ( {F}_{o} ( t ) - {F}_{m} ( t ) ) - {Q}_{o} ( t + Δ t ) ( ( {F}_{o} ( t + Δ t ) - {F}_{m} ( t + Δ t ) )} \qquad ( 4 )$$

Where

Fₘ(t), fraction of the gas component measured in the patient composite gas outflow at time t

Fₘ(t), fraction of the gas component measured in the patient composite gas outflow at time t

Qₒ(t), flow rate of the apparatus gas flow provided to the patient (apparatus gas flow rate), at time t

Fₘ(t+Δt), fraction of the gas component measured in the patient composite gas outflow (e.g. this is the measured CO₂, O₂, Nitrogen, Helium and/or Anaesthetic agent such as sevoflurane fraction parameter of the composite gas outflow) at time t+Δt

Qₒ(t+Δt), flow rate of the apparatus gas flow provided to the patient (apparatus gas flow flow rate) at time t+Δt

Fₒ(t), volume fraction of the gas component in the apparatus gas flow 11′ coming from the respiratory apparatus, at time t and t+Δt.

Fₒ(t+Δt), volume fraction of the gas component in the apparatus gas flow 11′ coming from the respiratory apparatus, at time t+Δt.

Qₒ is the flow rate of the apparatus gas flow

Fₒ is the fraction of the gas component in the apparatus gas flow

F_{E} is the fraction of the gas component in the exhaled patient gas flow

Optionally the parameter of gas present in an exhaled gas flow is gas fraction and determining the fraction of the gas (F_{E}) present in an exhaled gas flow using the determined parameter of gas present in the composite gas outflow and the time-varying flow rate comprises determining the gas fraction F_{E}(t) as a function of:

Qₒ(t),Qₒ(t+Δt),Fₘ(t+Δt),Fₘ(t)

where

Fₘ(t), volume fraction of the gas component measured in the patient composite gas outflow 15′ from the patient (this is the measured CO₂/O₂ fraction parameter of the composite gas outflow 15′, preferably measured by the sensor 14) at time t.

Fₘ(t) is preferably measured at the mouth of the patient when the patient's mouth is open and/or the nose if the patient's mouth is closed.

Qₒ(t), flow rate of the apparatus gas flow 11′ provided from the respiratory apparatus to the patient (apparatus gas flow rate) at time t.

Fₘ(t+Δt), volume fraction of the gas component measured in the patient composite gas outflow 15′ (this is the measured CO₂/O₂ fraction parameter of the composite gas outflow, preferably measured by the sensor 14) at time t+Δt. Fₘ(t+Δt) is preferably measured at the mouth of the patient.

Qₒ(t+Δt), flow rate of apparatus gas flow 11′ provided from the respiratory apparatus to the patient (apparatus gas flow rate), at time t+Δt.

Optionally the parameter of the gas present in the exhaled gas flow is gas fraction and the gas is preferably CO₂ and determining the gas fraction (F_{E}) in the exhaled gas flow using the determined parameter of gas present in the composite gas outflow and the time-varying flow rate comprises using:

$${F}_{E} ( t ) ∼ {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{o} ( t ) {F}_{m} ( t )} \qquad ( 5 )$$

Where

F_{E}(t) is the CO₂ and/or O₂ concentration in the exhaled patient gas flow (volume fraction of expired gas)

Fₘ(t), fraction of CO₂ measured in the composite gas outflow at time t

Qₒ(t), flow rate of the apparatus gas flow provided to the patient (apparatus gas flow flow rate) at time t

Fₘ(t+Δt), fraction of CO₂ measured in the composite gas outflow at time t+Δt

Qₒ(t+Δt), flow rate of the apparatus gas flow provided to the patient (apparatus gas flow flow rate) at time t+Δt

Qₒ is the flow rate of the apparatus gas flow

F_{E} is the fraction of the gas in the exhaled gas flow

Optionally the parameter of the gas present in the composite gas outflow from the patient is measured at or near the mouth and/or nose of the patient.

Optionally the first and second flow rates are different flow rates.

Optionally the first and second flow rates are high flow rates.

Optionally the first and second flow rates are greater than or equal to about 0 L per minute, and preferably about or greater than about 20 L per minute, and more preferably between about 20 L per minute to about 90 L per minute.

Optionally the time varying flow rate is an oscillation with a varying flow rate of great than or equal to about 0 L per minute, and preferably about or greater than about 20 L per minute, and more preferably between about 20 L per minute to about 90 L per minute.

Optionally the method comprises providing the apparatus gas flow during an anaesthetic procedure.

Optionally the apparatus gas flow is provided via a non-sealing patient interface, preferably a non-sealing cannula.

Optionally the apparatus gas flow is a high flow gas flow.

Optionally the method further comprises humidifying the apparatus gas flow.

Optionally determining the parameter of the gas present in the exhaled gas flow using the measured parameter of the gas present in the composite gas outflow comprising measuring just one gas and just the time varying parameter, the time varying parameter being flow rate.

In another aspect the present invention may be said to comprise a method of determining a parameter of a gas present in an exhaled gas flow comprising: providing an apparatus gas flow with a time-varying gas proportion (for example gas fraction) to a patient, determining a parameter of the gas present in a composite gas outflow from the patient, the patient composite gas outflow comprising: leak gas flow from apparatus gas flow, and exhaled gas flow from a patient with the gas, and determining the parameter of the gas present in the exhaled gas flow using the determined parameter of the gas present in the composite gas outflow and the time-varying gas proportion (for example gas fraction).

Optionally the apparatus gas flow with the time-varying gas fraction comprises at least a first gas fraction at a first time and a second gas fraction at a second time, and determining the parameter of the gas present in the exhaled gas flow using the determined parameter of the gas present in the composite gas outflow and the time-varying gas fraction comprises: using the determined parameter of the gas present in the composite outflow determined at the first gas fraction and determined at the second gas fraction.

Optionally the parameter comprises a fraction of the gas component in the exhaled gas flow.

Optionally the gas is: CO₂, O₂, Nitrogen, and/or Helium, Anaesthetic agent such as sevoflurane.

Optionally the parameter of the gas present in the composite gas outflow is determined during an inspiration and/or expiration phase of the patient breathing.

Optionally the parameter of gas present in an exhaled gas flow is gas fraction and determining the fraction of the gas present (F_{E}) in an exhaled gas flow using the determined parameter of gas present in the composite gas outflow and the time-varying gas fraction F_{E}(t) comprises determining the gas fraction as a function of:

Fₒ(t),Fₒ(t+Δt),Fₘ(t+Δt),Fₘ(t)

F_{E}(t) is the CO₂ and/or O₂ gas fraction in the exhaled patient gas flow (volume fraction of expired gas) at time t

Fₘ(t), fraction of CO₂ measured in the composite gas outflow at time t

Fₒ(t), gas fraction of the apparatus gas flow provided to the patient (apparatus gas flow gas fraction) at time t

Fₘ(t+Δt), fraction of CO₂ measured in the composite gas outflow at time t+Δt

Fₒ(t+Δt), gas fraction of the apparatus gas flow provided to the patient (apparatus gas flow gas fraction) at time t+Δt

Qₒ is the flow rate of the apparatus gas flow

F_{E} is the fraction of the gas in the exhaled gas flow

Optionally the parameter of the gas present in the exhaled gas flow is gas fraction and the gas is preferably CO₂, O₂, Nitrogen, Helium and/or Anaesthetic agent such as sevoflurane and determining the gas fraction in the exhaled gas flow using the determined parameter of gas present in the composite gas outflow and the time-varying gas fraction comprises using:

$${F}_{E} ( t ) = \frac{{F}_{m} ( t + Δ t ) ( {F}_{o} ( t ) - {F}_{m} ( t ) ) - {F}_{m} ( t ) ( {F}_{o} ( t + Δ t ) - {F}_{m} ( t + Δ t ) )}{( {F}_{o} ( t ) - {F}_{m} ( t ) ) - ( ( {F}_{o} ( t + Δ t ) - {F}_{m} ( t + Δ t ) )} \qquad ( 6 )$$

Where

F_{E}(t) is the CO₂ and/or O₂ concentration in the exhaled patient gas flow (volume fraction of expired gas)

Fₘ(t), fraction of CO₂ and/or O₂ or other gas measured in the composite gas outflow at time t

Fₒ(t), gas fraction of the apparatus gas flow provided to the patient (apparatus gas flow gas fraction) at time t

Fₘ(t+Δt), fraction of CO₂ and/or O₂ or other gas measured in the composite gas outflow at time t+Δt

Fₒ(t+Δt), gas fraction of the apparatus gas flow provided to the patient (apparatus gas flow gas fraction) at time t+Δt

Qₒ is the flow rate of the apparatus gas flow

F_{E} is the fraction of the gas in the exhaled gas flow

Optionally the parameter of the gas present in the composite gas outflow from the patient is measured at or near the mouth and/or nose of the patient.

Optionally the first and second gas fractions are different gas fractions.

Optionally the gas is O₂, and the method further comprises determining the CO₂ proportion present in the exhaled gas flow using the determined O₂ proportion and a function of:

F_{mCO2},k,Qₒ,Q_{E}

where

F_{mCO2} is the fraction of CO₂ in the patient composite gas outflow from the patient

k is the proportion of the apparatus gas flow that comes out through the patient's mouth (and (1−k) is the proportion that goes through the nose)

Qₒ is the flow rate of the apparatus gas flow.

Q_{E} is the flow rate of the patient exhaled gas flow.

Optionally the gas is O₂, and the method further comprises determining the CO₂ proportion present in the exhaled gas flow using the determined O₂proportion and

$${F}_{ECO 2} ( t ) = {F}_{mCO 2} ( t ) [ 1 + \frac{{F}_{mO 2} ( t ) - {F}_{EO 2} ( t )}{{F}_{oO 2} ( t ) - {F}_{mO 2} ( t )} ] \qquad ( 7.9 )$$

where

F_{mCO2} is the fraction of CO₂ in the patient composite gas outflow from the patient.

k is the proportion of the apparatus gas flow that comes out through the patient's mouth (and (1−k) is the proportion that goes through the nose)

Qₒ is the flow rate of the apparatus gas flow.

Q_{E} is the flow rate of the patient exhaled gas flow.

Optionally, the gas is O₂, and the method further comprises determining the CO₂ proportion present in the exhaled gas flow using the determined O₂ proportion and a function of:

F_{mCO2},F_{mO2},F_{EO2},F_{oO2}

where

F_{mCO2} is the fraction of CO₂ measured in the patient composite gas outflow from the patient

F_{mO2} is fraction of O₂ measured in the patient composite gas outflow from the patient

F_{EO2} is the fraction of O2 in the exhaled patient gas flow

F_{oO2} is the fraction of O2 in the apparatus gas flow provided from the respiratory apparatus to the patient.

Optionally the gas is O₂, and the method further comprises determining the CO₂ proportion present in the exhaled gas flow using the determined O₂ proportion and

$${F}_{ECO 2} ( t ) = {F}_{mCO 2} ( t ) [ 1 + \frac{{F}_{mO 2} ( t ) - {F}_{EO 2} ( t )}{{F}_{oO 2} ( t ) - {F}_{mO 2} ( t )} ] \qquad ( 7.9 )$$

where at time t,

F_{mCO2} is the fraction of CO₂ measured in the patient composite gas outflow from the patient

F_{mO2} is fraction of O₂ measured in the patient composite gas outflow from the patient

F_{EO2} is the fraction of O2 in the exhaled patient gas flow

Fₒ₀₂ is the fraction of O2 in the apparatus gas flow provided from the respiratory apparatus to the patient.

In another aspect the present invention may be said to comprise an apparatus for providing an apparatus gas flow and determining a parameter of gas present in an exhaled patient gas flow, comprising: a flow source, a sensor for sensing a composite gas outflow, and a controller, wherein the apparatus is configured to: provide an apparatus gas flow with a time-varying flow rate, determine a parameter of a gas present in a composite gas outflow from the patient, the composite gas outflow comprising: leak gas flow from apparatus gas flow, and exhaled gas flow from a patient with the gas, and determine the parameter of the gas present in the exhaled gas flow using the determined parameter of gas present in the composite gas outflow and the time-varying flow rate.

Optionally the apparatus further comprises a humidifier for humidifying the apparatus gas flow.

Optionally the apparatus further comprises a non-sealing patient interface, and preferably a non-sealing nasal cannula to provide the apparatus gas flow to a patient.

Optionally the apparatus gas flow is a high flow gas flow.

Optionally the apparatus gas flow with the time-varying flow rate comprises at least a first flow rate at a first time and a second flow rate at a second time, and determining the parameter of the gas present in the exhaled gas flow using the determined parameter of the gas present in the composite gas outflow and the time-varying flow rate comprises: using the determined parameter of the gas present in the composite outflow determined at the first flow rate and determined at the second flow rate.

Optionally the parameter comprises a fraction of the gas component in the exhaled gas flow.

Optionally the gas is: CO₂, O₂, Nitrogen, Helium and/or Anaesthetic agent such as sevoflurane and/or the sensor is configured to sense one or more of the following in the composite gas outflow: CO₂, O₂, Nitrogen, Helium, and/or anaesthetic agent such as sevoflurane.

Optionally the parameter of gas present in an exhaled gas flow is gas fraction and determining the fraction of the gas (F_{E}) present in an exhaled gas flow using the determined parameter of gas present in the composite gas outflow and the time-varying flow rate comprises determining the gas fraction as a function of:

Qₒ(t),Qₒ(t+Δt),Fₘ(t+Δt),Fₘ(t)

Where

F_{E}(t) is the gas component concentration in the exhaled patient gas flow (volume fraction of expired gas)

Fₘ(t), fraction of the gas component measured in the patient composite gas outflow at time t

Qₒ(t), flow rate of the apparatus gas flow provided to the patient (apparatus gas flow rate), at time t

Fₘ(t+Δt), fraction of the gas component measured in the patient composite gas outflow (this is the measured CO₂/O₂ fraction parameter of the composite gas outflow) at time t+Δt

Qₒ(t+Δt), flow rate of the apparatus gas flow provided to the patient (apparatus gas flow flow rate) at time t+Δt

Optionally the parameter of the gas present in the exhaled gas flow is gas fraction and the gas is preferably CO₂ and determining the gas fraction in the exhaled gas flow using the determined parameter of gas (F_{E}) present in the composite gas outflow and the time-varying flow rate comprises using:

$${F}_{E} ( t ) ∼ {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{o} ( t ) {F}_{m} ( t )} \qquad ( 5 )$$

Where

F_{E}(t) is the CO₂ or O₂or other gas fraction in the exhaled patient gas flow (volume fraction of expired gas)

Fₘ(t), fraction of CO₂ measured in the composite gas outflow at time t

Qₒ(t), flow rate of the apparatus gas flow provided to the patient (apparatus gas flow flow rate) at time t

Fₘ(t+Δt), fraction of CO₂ measured in the composite gas outflow at time t+Δt Qₒ(t+Δt), flow rate of the apparatus gas flow provided to the patient (apparatus gas flow flow rate) at time t+Δt

Qₒ is the flow rate of the apparatus gas flow

F_{E} is the fraction of the gas in the exhaled gas flow

Optionally the sensor is positioned to measure the parameter of the gas present in the composite gas outflow from the patient at or near the mouth and/or nose of the patient.

In another aspect the present invention may be said to comprise an apparatus for providing an apparatus gas flow and determining a parameter of gas present in an exhaled patient gas flow, comprising: a flow source, a sensor for sensing a composite gas outflow, and a controller, wherein the apparatus is configured to: provide an apparatus gas flow with a time-varying gas proportion (for example gas fraction), determine a parameter of a gas present in a composite gas outflow from the patient, the composite gas outflow comprising: leak gas flow from apparatus gas flow, and exhaled gas flow from a patient with the gas, and determine the parameter of the gas present in the exhaled gas flow using the determined parameter of gas present in the composite gas outflow and the time-varying gas proportion (for example gas fraction).

Optionally the apparatus gas flow with the time-varying gas fraction comprises at least a first gas fraction at a first time and a second gas fraction at a second time, and determining the parameter of the gas present in the exhaled gas flow using the determined parameter of the gas present in the composite gas outflow and the time-varying gas fraction comprises: using the determined parameter of the gas present in the composite outflow determined at the first gas fraction and determined at the second gas fraction.

Optionally the parameter comprises a fraction of the gas component in the exhaled gas flow.

Optionally the gas is: CO₂, O₂, Nitrogen, Helium, and/or Anaesthetic agent such as sevoflurane.

Optionally the parameter of gas present in an exhaled gas flow is gas fraction and determining the fraction of the gas (F_{E}) present in an exhaled gas flow using the determined parameter of gas present in the composite gas outflow and the time-varying gas fraction comprises determining the gas fraction as a function of:

F,Fₒ(t+Δt),Fₘ(t+Δt),Fₘ(t)

Where

F_{E}(t) is the gas component concentration in the exhaled patient gas flow (volume fraction of expired gas)

Fₘ(t), fraction of the gas component measured in the patient composite gas outflow at time t

Fₒ(t), gas fraction of the apparatus gas flow provided to the patient (apparatus gas flow gas fraction), at time t

Fₘ(t+Δt), fraction of the gas component measured in the patient composite gas outflow (this is the measured CO₂/O₂ fraction parameter of the composite gas outflow) at time t+Δt

Fₒ(t+Δt), gas fraction of the apparatus gas flow provided to the patient (apparatus gas flow gas fraction) at time t+Δt.

Optionally the parameter of the gas present in the exhaled gas flow is gas fraction and the gas is preferably CO₂, O₂, Nitrogen, Helium and/or Anaesthetic agent such as sevoflurane and determining the gas fraction (F_{E}) in the exhaled gas flow using the determined parameter of gas present in the composite gas outflow and the time-varying gas fraction comprises using:

$${F}_{E} ( t ) = \frac{{F}_{m} ( t + Δ t ) ( {F}_{o} ( t ) - {F}_{m} ( t ) ) - {F}_{m} ( t ) {F}_{o} ( t + Δ t ) - {F}_{m} ( t + Δ t ) )}{( {F}_{o} ( t ) - {F}_{m} ( t ) ) - ( ( {F}_{o} ( t + Δ t ) - {F}_{m} ( t + Δ t ) )} \qquad ( 6 )$$

Where

F_{E}(t) is the CO₂ or O₂ or other gas in the exhaled patient gas flow (volume fraction of expired gas) at time t

Fₘ(t), fraction of CO₂ measured in the composite gas outflow at time t

Fₒ(t), gas fraction of the apparatus gas flow provided to the patient (apparatus gas flow gas fraction) at time t

Fₘ(t+Δt), fraction of CO₂ measured in the composite gas outflow at time t+Δt

Fₒ(t+Δt), gas fraction of the apparatus gas flow provided to the patient (apparatus gas flow gas fraction) at time t+Δt

Qₒ is the flow rate of the apparatus gas flow

F_{E} is the fraction of the gas in the exhaled gas flow

Optionally the sensor is positioned to measure the parameter of the gas present in the composite gas outflow from the patient at or near the mouth and/or nose of the patient.

In another aspect the present invention may be said to consist in a method of determining a O₂ and/or CO₂ fraction present in an exhaled gas flow comprising: providing a humidified high flow apparatus gas flow with a time-varying flow rate to a patient via a non-sealing nasal cannula, measuring a fraction of O₂ and/or CO₂ present in a composite gas outflow from the patient, and determining the fraction O₂ and/or CO₂ present in the exhaled gas flow using the measured fraction of O₂ or fraction of CO₂ present in the composite gas outflow and the time-varying flow rate.

In another aspect the present invention may be said to comprise a non-transitory computer-readable medium having stored thereon computer executable instructions that, when executed on a processing device or devices, cause the processing device or devices to perform a method of determining a parameter of a gas present in an exhaled gas flow comprising: providing an apparatus gas flow with a time-varying parameter to a patient, measuring a parameter of the gas present in a composite gas outflow from the patient, and determining the parameter of the gas present in the exhaled gas flow using the measured parameter of the gas present in the composite gas outflow and the time-varying parameter.

In another aspect the present invention may be said to comprise a method of determining a parameter of an exhaled patient gas flow comprising: providing an apparatus gas flow with a time-varying parameter to a patient, determining a parameter of a composite gas outflow from the patient, the composite gas outflow comprising: a leak gas flow from the apparatus gas flow, and the exhaled patient gas flow with a gas component, and the determined parameter of the composite gas outflow being the proportion of the gas component in the composite gas outflow, and determining a proportion of the gas present in the exhaled gas flow using the determined parameter of the composite gas outflow and the time-varying parameter.

Optionally the sensor is senses composite gas flow by sensing gas flow the patient's:
mouth and nose,
mouth, or
nose.

In another aspect the present invention may be said to comprise a method of determining CO₂ fraction present in an exhaled gas flow comprising: providing a high flow apparatus gas flow to a patient, determining a fraction of O₂ in a patient exhaled gas flow, and determining the CO₂ proportion present in the exhaled gas flow using the determined O₂ proportion and a function of:

FₘCO₂,k,Qₒ,Q_{E}

where FₘCO₂ is the volume fraction of CO₂ of the apparatus gas flow.

k is the proportion of the apparatus gas flow that comes out through the patient's mouth (and (1−k) is the proportion that goes through the nose)

Qₒ is the flow rate of the apparatus gas flow.

Q_{E} is the flow rate of the patient exhaled gas flow.

In another aspect the present invention may be said to comprise a method of determining CO₂ fraction present in an exhaled gas flow comprising: providing a high flow apparatus gas flow to a patient, determining a fraction of O₂ in a patient exhaled gas flow, and determining the CO₂ proportion present in the exhaled gas flow using the determined O₂ proportion and

$${F}_{ECO 2} = {F}_{mCO 2} ( 1 + \frac{k {Q}_{o}}{{Q}_{E}} ) \qquad ( 8 )$$

where FₘCO₂ is the volume fraction of CO₂ of the apparatus gas flow.

k is the proportion of the apparatus gas flow that comes out through the patient's mouth (and (1−k) is the proportion that goes through the nose)

Qₒ is the flow rate of the apparatus gas flow.

Q_{E} is the flow rate of the patient exhaled gas flow.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner. Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The phrase ‘computer-readable medium’ should be taken to include a single medium or multiple media. Examples of multiple media include a centralised or distributed database and/or associated caches. These multiple media store the one or more sets of computer executable instructions. The phrase ‘computer readable medium’ should also be taken to include any medium that is capable of storing, encoding or carrying a set of instructions for execution by a processor of a computing device and that cause the processor to perform any one or more of the methods described herein. The computer-readable medium is also capable of storing, encoding or carrying data structures used by or associated with these sets of instructions. The phrase ‘computer-readable medium’ includes solid-state memories, optical media and magnetic media.

In this specification where reference has been made to patent specifications, other external documents, or other sources of information, this is generally for the purpose of providing a context for discussing the features of the disclosure. Unless specifically stated otherwise, reference to such external documents is not to be construed as an admission that such documents, or such sources of information, in any jurisdiction, are prior art, or form part of the common general knowledge in the art.

The invention may also be said broadly to consist in the parts, elements and features referred to or indicated in the specification of the application, individually or collectively, in any or all combinations of two or more of said parts, elements or features. Where, in the foregoing description reference has been made to integers or components having known equivalents thereof, those integers are herein incorporated as if individually set forth.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting. Where specific integers are mentioned herein which have known equivalents in the art to which this invention relates, such known equivalents are deemed to be incorporated herein as if individually set forth. The invention consists in the foregoing and also envisages constructions of which the following gives examples only.

### BRIEF DESCRIPTION OF DRAWINGS

Embodiments will be described with reference to the following drawings, of which:

FIG. 1A shows flows between a respiratory apparatus, a patient and the patient's environment.

FIG. 1B is a respiratory apparatus for providing high flow.

FIG. 2 is a trace of CO2 fraction in a composite and exhaled gas flow

FIG. 3A shows components of an apparatus gas flow (time-varying flow rate) and a resultant composite gas outflow.

FIGS. 3B and 3C show alternative apparatus gas flows.

FIG. 4 is an embodiment of a respiratory apparatus which implements time-varying apparatus flow and estimates exhaled gas parameters.

FIG. 5 is an embodiment of a method implemented by the respiratory apparatus for time-varying flow rate gas flow and estimating exhaled gas parameters.

FIG. 6A shows components of an apparatus gas flow (time-varying gas fraction) and a resultant composite gas outflow.

FIGS. 6B and 6C show alternative apparatus gas flows.

FIG. 7 is an embodiment of a method implemented by the respiratory apparatus for time-varying gas fraction apparatus gas flow and estimating exhaled gas parameters.

FIG. 8 shows the various gas flows into and out of the patient.

### DETAILED DESCRIPTION

### 1. Overview

The present embodiments relate to determining a gas parameter in a gas flow exhaled (“exhaled gas flow”) by a patient when using a respiratory apparatus providing (preferably high) flows through a non-sealing patient interface, e.g. a non-sealing nasal cannula. (“Determining a gas parameter” can mean, without limitation, determining, gaining or otherwise obtaining an estimate, value, indication or some other information of or about the gas parameter)

The embodiments described provide an apparatus and method for determining a parameter of an exhaled gas flow by a patient, the parameter relating to the proportion (e.g. concentration/fraction or partial pressure) of a gas component in the exhaled gas flow comprising two or more constituent gas components. In certain situations, the patient is spontaneously breathing (that is, breathing under their own effort, even if it is shallow or otherwise compromised breathing). For example, the exhaled gas flow might comprise O₂, CO₂, Nitrogen, Helium, an anaesthetic agent (such as sevoflurane) etc., and the parameter might be the proportion (e.g. concentration/fraction or partial pressure) of CO₂ or the proportion (e.g. concentration/fraction or partial pressure) of O₂ in the gas flow exhaled from a patient. Here, the gas component is CO₂ or O₂, and the parameter is the proportion that gas component makes up of the exhaled gas flow. In some embodiments, the parameter may related to a gas other than CO₂ or O₂.

A medical professional might want to gain an estimate of a parameter of exhaled gas flow when monitoring a patient for example during a medical procedure. Medical procedure should be considered broadly and can comprise any aspect of providing a medical procedure, comprising operative procedures, pre and post-operative procedures, any time prior to, during or after sedation or anaesthesia (sedation and anaesthesia more generally referred to herein as “anaesthetic procedures”), including administering sedatives and/or anaesthetics, during oxygenation and pre-oxygenation phases or procedures, or at any other time without limitation. Medical procedure can also encompass providing respiratory support such as high flow respiratory support. In the context of this specification, medical procedure can also encompass monitoring a patient, whether or not a particular procedure is being provided to the patient. The embodiments described are not just restricted to use in medical procedures. It could be used in ICU, or any other situation where respiratory support is provided.

In this specification, reference to “exhale” can be used interchangeably with “expire”

In this specification, reference to “proportion” in the context of gas refers to any relative measure of a constituent gas component in a total gas comprising two or more constituent gas components. For example, proportion could cover:
volume fraction,
fraction,
volume concentration,
concentration,
molarity,
partial pressure

The proportion measured may be the parameter that is measured by the sensor being used, be it concentration, fraction, partial pressure or otherwise. The proportion determined may be the parameter desired by a user and/or processed by a component of a respiratory system or associated with a respiratory system.

In this specification, reference to “concentration” can also be termed “fraction” and can be indicated as percentage by volume of the gas of interest versus the volume of constituent gases overall in the gas flow in question, be it exhaled gas flow, apparatus flow or any other flow. However, the parameter could be a different measure and the gas could be different—these are just examples.

The gas relating to the gas parameter being determined could be, without limitation, oxygen (O₂), carbon dioxide (CO₂), nitrogen (N), helium (He) or Sevoflurane. Where reference is made to a particular gas herein, it will be appreciated that it is by way of example only and the description can apply to any gas—not just that referenced.

In this specification, “high flow” means, without limitation, any gas flow with a flow rate that is higher than usual/normal, such as higher than the normal inspiration flow rate of a healthy patient. It can be provided by a non-sealing respiratory system with substantial leak happening at the entrance of the patient's airways due to a non-sealing patient interface, for example non-sealing prongs. It is also provided with humidification to improve patient comfort, compliance and safety. Alternatively or additionally, it can be higher than some other threshold flow rate that is relevant to the context—for example, where providing a gas flow to a patient at a flow rate to meet inspiratory demand, that flow rate might be deemed “high flow” as it is higher than a nominal flow rate that might have otherwise been provided. “High flow” is therefore context dependent, and what constitutes “high flow” depends on many factors such as the health state of the patient, type of procedure/therapy/support being provided, the nature of the patient (big, small, adult child) and the like. Those skilled in the art know from context what constitutes “high flow”. It is a magnitude of flow rate that is over and above a flow rate that might otherwise be provided.

But, without limitation, some indicative values of high flow can be as follows.
In some configurations, delivery of gases to a patient at a flow rate of greater than or equal to about 5 or 10 litres per minute (5 or 10 LPM or L/min).
In some configurations, delivery of gases to a patient at a flow rate of about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied or provided to an interface via a system or from a flow source, may comprise, but is not limited to, flows of at least about 5, 10, 20, 30, 40, 50, 60, 70, 80, 90, 100, 110, 120, 130, 140, 150 LPM, or more, and useful ranges may be selected to be any of these values (for example, about 20 LPM to about 90 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

In “high flow” the gas delivered will be chosen depending on for example the intended use of a therapy. Gases delivered may comprise a percentage of oxygen. In some configurations, the percentage of oxygen in the gases delivered may be about 15% to about 100%, about 20% to about 100%, or about 21% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

In some embodiments, gases delivered may comprise a percentage of carbon dioxide. In some configurations, the percentage of carbon dioxide in the gases delivered may be more than 0%, about 0.3% to about 100%, about 1% to about 100%, about 5% to about 100%, about 10% to about 100%, about 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

Flow rates for “High flow” for premature/infants/paediatrics (with body mass in the range of about 1 to about 30 kg) can be different. The therapeutic flow rate can be set to 0.4-0.8 L/min/kg with a minimum of about 0.5 L/min and a maximum of about 25 L/min. For patients under 2 kg maximum flow is set to 8 L/min.

The oscillating flow is set to 0.05-2 L/min/kg with a preferred range of 0.1-1 L/min/kg and another preferred range of 0.2-0.8 L/min/kg.

The therapeutic flow rate can be time-varying (e.g. oscillating)—that is, the therapeutic flow can have a time-varying (e.g. oscillating) flow rate component. This time-varying flow rate flow rate can help with therapy.

Note, the embodiments herein also have signature flow rates that are time-varying (e.g. oscillate) and might be in addition to the therapeutic flow rates. Therefore, where therapeutic time-varying flow rates are used, the gas flow rate from the apparatus will have a therapeutic time-varying gas flow component(s) (portion) and a signature time-varying flow rate component(s) (portion). Therapeutic time-varying flow rates have a different purpose to signature time-varying flow rates, and might be different frequencies and/or amplitudes (although, they could overlap or be the same). Signature flow rates could be lower, the same or higher than therapeutic flow rates. Signature flow rate frequencies could be lower, the same or higher than therapeutic flow rate frequencies (when time-varying). In some embodiments, the signature flow rate has a higher frequency that the therapeutic flow rate. Therapeutic time-varying flow rates are to provide respiratory support, airway clearance, oxygenation or the like whereas signature time-varying flow rates are to assist with determining a gas parameter. Signature time-varying flow rates will be described in more detail later. Throughout the specification, unless otherwise stated, the focus will be on the signature time-varying flow rates, but that doesn't exclude that there might also be therapeutic time-varying flow rates for therapeutic reasons.

As an example, the signature flow rates can step between a first flow rate and second flow rate, one or both of which can fall in the range of about 0 LPM to 70 LPM. The maximum signature flow rate could be the therapeutic flow rate. The signature flow rate could be combined with the therapeutic flow rate (e.g. added) or it could form part or all of the therapeutic flow rate—that is, the therapeutic flow rate itself could be the signature flow rate. In some embodiments, the signature flow rate can be related to the therapeutic flow rate as a percentage. For example, the signature time-varying flow rate (adults) is in the range of:
about 0% to about 200% of the therapeutic flow rate,
about 0% to 100% of the therapeutic flow rate,
about 100% to 200% of the therapeutic flow rate, or
about 50% to 150% of the therapeutic flow rate,
   and/or is in the range of
about 0-140 LPM,
about 0-70 LPM,
about 70-140 LPM,
about 40-100 LPM, or
about 20-60 LPM

These are not limiting flow rates, and it should also be noted that the signature flow rates could be negative, but when combined with the therapeutic flow rates create a total flow rate that is positive.

High flow has been found effective in meeting or exceeding the patient's normal real inspiratory flow, to increase oxygenation of the patient and/or reduce the work of breathing. Additionally, high flow may generate a flushing effect in the nasopharynx such that the anatomical dead space of the upper airways is flushed by the high incoming gas flows. This creates a reservoir of fresh gas available of each and every breath, while minimising re-breathing of carbon dioxide, nitrogen, etc.

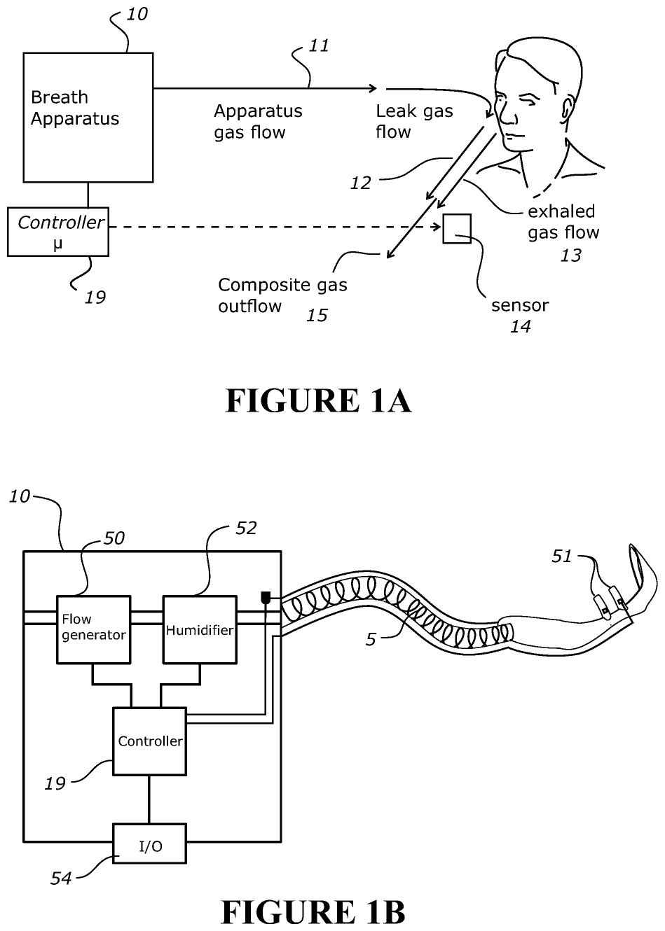

By way of example, a high flow respiratory apparatus 10 is described with reference to e.g. FIG. 1A and 1B. In general terms, the apparatus comprises a main housing 10 that contains a flow generator 50 in the form of a motor/impeller arrangement, an optional humidifier 52, a controller 19, and a user I/O interface (comprising, for example, a display and input device(s) such as button(s), a touch screen, or the like). The controller 19 is configured or programmed to control the components of the apparatus, including: operating the flow generator to create a flow of gas (gas flow) for delivery to a patient, operating the humidifier (if present) to humidify and/or heat the generated gas flow, receive user input from the user interface for reconfiguration and/or user-defined operation of the apparatus, and output information (for example on the display) to the user. The user could be a patient, healthcare professional, or anyone else interested in using the apparatus. A patient breathing conduit is coupled to a gas flow output in the housing of the flow therapy apparatus, and is coupled to a patient interface 51 such as a nasal cannula with a manifold and nasal prongs. The patient breathing conduit can have a heater wire 5 to heat gas flow passing through to the patient.

High flow may be used as a means to promote gas exchange and/or respiratory support through the delivery of oxygen and/or other gases, and through the removal of CO₂ from the patient's airways. High flow may be particularly useful prior to, during or after a medical procedure.

Further advantages of high gas flow can comprise that the high gas flow increases pressure in the airways of the patient, thereby providing pressure support that opens airways, the trachea, lungs/alveolar and bronchioles. The opening of these structures enhances oxygenation, and to some extent assists in removal of CO₂.

The increased pressure can also keep structures such as the larynx from blocking the view of the vocal chords during intubation. When humidified, the high gas flow can also prevent airways from drying out, mitigating mucociliary damage, and reducing risk of laryngospasms and risks associated with airway drying such as nose bleeding, aspiration (as a result of nose bleeding), and airway obstruction, swelling and bleeding. Another advantage of high gas flow is that the flow can clear smoke created during surgery in the air passages. For example, smoke can be created by lasers and/or cauterizing devices.

Referring to FIG. 1A, the present embodiments can be used in any suitable situation where a patient is provided a gas flow from a respiratory apparatus 10 for providing therapy (such as, a high gas flow for, but not limited to, high flow therapy). The apparatus 10 provides an apparatus gas flow 11. This apparatus gas flow 11 has a flow rate. The flow rate might be a constant flow rate (that is not varying over time), or it might be time-varying, depending on the requirements of therapy. In these situations, the patient will breathe at least some of the apparatus gas flow 11 and will exhale gas flow 13, and that exhaled gas flow 13 will have constituent gas components, such as CO₂, O₂, Nitrogen, Helium and the like. The exhaled gas flow 13 may also comprise anaesthetic agents, such as sevoflurane.

It would be useful to medical professionals to determine (e.g. measured through a sensor 14 or the like) the exhaled patient gas flow 13 when monitoring the patient. In particular, it would be useful to medical professionals to determine a parameter of the constituent gas components of the patient exhaled gas flow 13, such as the proportion (e.g. fraction) of CO₂ or O₂.This helps assess how the patient is responding to therapy, their general well-being and/or when a next stage of a medical procedure may commence if the patient is undergoing a medical procedure. For example, when pre-oxygenating a patient, a measure of the O₂ fraction in the exhaled gas flow 13 helps determine if pre-oxygenation has been achieved. As another example, measuring the CO₂ fraction helps determine if a patient is breathing. However, it can be difficult to determine the parameter of a gas component (be it O₂ fraction, CO₂ fraction or other gas parameter) of the exhaled gas flow 13 when an apparatus gas flow 11 is provided to the patient, because the leakage (“leak gas flow”) 12 from the apparatus gas flow 11 from the respiratory apparatus 10 adds to the exhaled gas flow 13 to create a total gas outflow (“composite gas outflow”) 15 from the patient that is measured by the e.g. sensor 14.

“Leak gas flow” 12 comprises the excess gas flow from the apparatus gas flow 11 that is not inhaled and/or has not entered the lower airways of the patient by the patient and escapes to ambient via the mouth and/or nose.

“Composite gas outflow” is the leak gas flow 12 combined with the exhaled gas flow 13. Therefore, the exhaled gas flow 13 is not actually measured, but rather a composite gas outflow 15 that includes the leak flow 12 combined with the patient's exhaled gas flow. The leak gas flow 12 can dilute (e.g. in the case of measuring CO₂ fraction) or increase (e.g. in the case of measuring O₂ fraction) or more generally “alter” the gas component of the exhaled gas flow 13 being measured by the sensor 14, giving misleading information about the parameter of the gas component being obtained. This issue is exacerbated at high flow rates, e.g. when providing high flow therapy. So, instead of measuring the exhaled gas flow 13, the sensor is actually measuring a gas component of the composite gas outflow 15 which comprises the exhaled gas flow 13 and possibly at least a portion of the apparatus gas flow 11 (that is, the leak gas flow 12). The exhaled gas flow 13 is not actually measured, but rather the composite gas outflow is measured, so the apparent reading of the exhaled gas flow is not accurate. Note, the composite gas outflow 15 can comprises other gases too—e.g. those present in ambient air.

Exhaled gas flow 13, leak gas flow 12 and the resulting composite gas out flow 15 can come out of the mouth, the nose or the mouth and/or nose. There are several scenarios: 1) A patient's mouth is open and the exhaled gas flow, the leak gas flow and therefore resulting composite gas outflow are predominantly (which can comprise entirely) coming out of the patient's mouth. 2) A patient's mouth is open and the exhaled gas flow, the leak gas flow and therefore resulting composite gas outflow are coming out of both the patient's mouth and nose. 3) A patient's mouth is closed and the exhaled gas flow, the leak gas flow and therefore resulting composite gas outflow are coming out of the patient's nose. When measuring the composite gas outflow 15, this can be done with a suitable sensor, either placed to measure flow out the mouth, flow out the nose, or flow out the nose and mouth. Where the sensor is measuring flow just out the mouth, or just out the nose, the sensor might not be measuring the entire composite gas outflow, as some might also be existing the other orifice (e.g. the other of the nose or mouth depending on which the sensor is not measuring). In this case, the sensor measurement is still suitable and/or obtaining enough of a measure of composite gas outflow for a determination of the gas parameter of the exhaled gas flow.

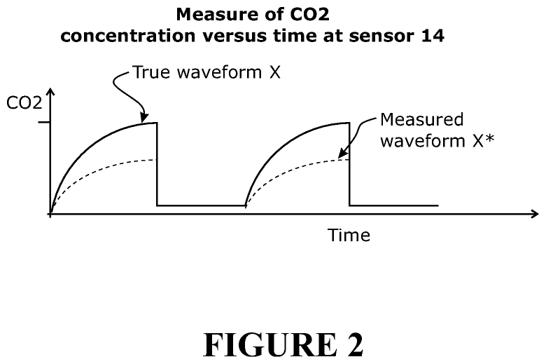

As an example, FIG. 2 shows, an exhaled CO₂ fraction measurement of the patient. FIG. 2 shows an exemplary carbon dioxide signal comparing the true CO₂ waveform (solid line) X to a measured, diluted waveform (dotted line) X*. It can be seen that in this setup both the magnitude and shape of the waveform that may be displayed to a medical professional is affected by the apparatus gas flow 11 dilution. While this information does provide an indication to a medical professional that gas exchange is occurring, it denies the medical professional further insights that may be gained from an accurate reading of the waveform magnitude and shape (for example from knowing the patient's end tidal CO₂, which is the level of carbon dioxide that is released by the patient at the end of an exhaled breath). This information may be useful to know during an anaesthetic procedure, such as procedural sedation, where the patient may be shallowly breathing. The actual measured waveform (which is effectively a measure of the composite gas outflow 15) is shown in dotted lines X*. However, this is a misleading waveform because the actual/true CO₂ proportion (in this case fraction) of the exhaled gas flow 13 is higher as shown in the solid line X. The measured waveform is lower because it actually measures the CO₂ fraction in the combination of the leak gas flow 12 and the exhaled gas flow 13 added together to form the composite gas outflow 15. The CO₂ fraction in the exhaled gas flow is actually much higher, but because of the contribution of gases from the leak gas-flow (which are lower in CO₂ fraction), the measured CO₂ fraction in the composite gas outflow is diluted. In some cases, the dilution may be so substantial that sometimes it may be difficult to detect a CO₂ signal at all. For example when the patient has shallow breathing and high flow rates are provided to the patient. Note, X* is an accurate measurement, but not a true reflection of the CO₂ fraction X of the exhaled gas flow 13′—rather it is a measure of the CO₂ fraction X* in the composite gas outflow 15′

A similar situation can be envisaged for measuring O₂ fraction in the exhaled gas flow 13. Where the O₂ fraction of the patient exhalation is less than the O₂ fraction provided by the respiratory apparatus, the leak gas flow 12 will increase the O₂ fraction of the composite gas outflow 15, thus giving a misleading representation of the actual O₂ fraction in the exhaled gas flow 13.

The present embodiments relate to an non-sealing respiratory apparatus that provides, preferably high, flows of gas to a patient. The non-sealing apparatus means some of the gas flow is not inhaled by the patient, but rather “leaks” (leak flow 12) to ambient. The embodiments provide an apparatus and method to determine an actual exhaled gas flow 13 parameter of a desired gas component by measuring a parameter of the gas component in a composite gas outflow 15 at or near (“proximate”) the patient, and taking into account the effect of the leak gas flow 12 in the measure of the parameter and adjusting the measure accordingly (or otherwise using the measure and other information) to determine the parameter of the desired gas component in the actual exhaled gas flow 13 from the patient. The apparatus gas flow can be varied with a signature to assist to determine the parameter. Note, the composite gas outflow 15 can comprise other gases too (in addition to CO₂ and O₂)—e.g. those present in ambient air. The present embodiments described work in the presence of such additional gases.

The respiratory apparatus can comprise a flow source that is able to provide apparatus gas flow to the patient. The apparatus provides a time-varying apparatus gas flow, such that a time-varying parameter of the apparatus gas flow varies over time. This provides a signature that can be used to assist determine a gas parameter of actual exhaled gas flow 13. As possible examples, the time-varying parameter of the apparatus gas flow might be flow rate, or a gas proportion (such as gas fraction (e.g. O₂ fraction) and/or gas partial pressure (e.g. O₂ partial pressure)).

In an embodiment, the flow source provides a time-varying apparatus gas flow that has a time-varying flow rate. In another embodiment, the flow source provides a time-varying apparatus gas flow that has a time-varying gas proportion (such as gas fraction or gas partial pressure). The flow source may be able to provide gas flows to the patient at two or more flow rates, or two or more gas proportions. The flow source may vary (e.g. oscillate, although not necessarily at a fixed frequency) the flow rate provided to the patient, for example between the two or more flow rates or between two or more gas proportions. In the case of varying between two or more gas proportions, preferably the flow rate is not varied with a signature flow rate—just the therapeutic component is provided without any non-therapeutic flow rate variation being applied.

The respiratory apparatus can comprise one or more sensors to measure a desired gas parameter at the two or more flow rates and/or two or more gas proportions and a controller to determine a parameter of an exhaled patient gas flow 13. Where a target gas is delivered to the patient, e.g. O₂, the respiratory apparatus may comprise an input to control the concentration/fraction or partial pressure of the target gas in the delivered gas flow. The input could be manual (e.g. flow meter dial) or electronic. Reference herein to a controller configured to carry out a function could also mean one or more controllers configured to carry out such function, and reference to controller should not be considered limiting to the physical apparatus used. A non-transitory computer readable medium storing a program to execute the method on the controller can be provided.

In one embodiment, a gas flow is provided to the patient at first and second gas flow rates—or a time-varying (e.g. oscillating) flow rate (either varying continuously or discretely to create a plurality of gas flow rates) is provided (which has at least a first and second gas flow rate). This is a signature time-varying gas flow rate. As noted above, the time-varying gas flow could also have a therapeutic time-varying gas flow rate portion (as well as a signature time-varying gas flow rate portion). For explanatory purposes, the embodiment here is described with reference to just the signature time-varying flow rate portion—but that doesn't exclude the possibility that the flow rate also has a therapeutic time-varying portion (or that therapeutic portion forms the dual purpose of signature time-varying portion also). A gas parameter (of the target gas) is then measured at the patient (that is, a gas parameter of the composite gas outflow 15) at the time of the first and the time of the second flow rates (or in the case of varying, at the time of a first and second gas flow rate of the plurality of gas flows). The first and second gas flow rates and the measured gas parameter at each time are used to determine a expired gas parameter (that is, gas parameter of the exhaled gas flow 13), e.g. using equation 4. This process can then be repeated over a period of time and the determined, expired gas parameter can be extrapolated and presented as a signal. It should be noted, that in embodiments, rather than the controller receiving measurements of the gas parameter directly from the sensor, it could be received indirectly via input on a user interface from a user.

Alternatively in another embodiment, a gas flow is provided to the patient at first and second gas proportions (e.g. the fraction of oxygen in the apparatus gas flow 11 may be varied) or a varying (e.g. oscillating) gas proportion (either varying continuously or discretely to create a plurality of gas proportions). A gas parameter (of the target gas) is then measured at the patient at the time of the first gas fraction and time of the second gas fraction (or in the case of varying, at the time of a first and second gas proportion of the plurality of gas proportions). The first and second gas fractions or a varying (e.g. oscillating) fraction is provided (which has at least a first and second gas fractions). A gas parameter is then measured at the first and second fractions and the measured gas parameters at each time are used to determine a expired gas parameter e.g. using equation 4. This process can then be repeated over a period of time and the determined, expired gas parameter can be extrapolated and presented as a signal. This approach is particularly suited to gases that are commonly administered to a patient (e.g. oxygen). It should be noted, that in embodiments, rather than the controller receiving measurements of the gas parameter directly from the sensor, it could be received indirectly via input on a user interface from a user.

Alternatively in another embodiment, a gas flow is provided to the patient at first and second gas proportions (e.g. the partial pressure of oxygen in the apparatus gas flow 11 may be varied). A gas parameter is then measured at the patient at the time of the first gas partial pressure and time of the second gas partial pressure. The first and second gas partial pressure or a varying (e.g. oscillating) partial pressure is provided (which has at least a first and second gas partial pressure). A gas parameter (of the target gas) is then measured at the first and second partial pressure and the measured gas parameters at each time are used to determine a expired gas parameter e.g. using equation 4. This process can then be repeated over a period of time and the determined, expired gas parameter can be extrapolated and presented as a signal. This approach is particularly suited to gases that are commonly administered to a patient (e.g. oxygen). It should be noted, that in embodiments, rather than the controller receiving measurements of the gas parameter directly from the sensor, it could be received indirectly via input on a user interface from a user.

Note, “at the time of” does not have to be temporarily exact, and can mean “about the time of” where any small difference in time does not alter the efficacy of the measurement. Also, it should be noted that when the apparatus changes the flow rate or gas proportion (e.g. gas fraction or gas partial pressure) of the gas flow, there might be a delay between the change in flow rate/gas proportion at the breathing apparatus 10, and that new flow rate/gas proportion reaching the patient due to a distance the gas flow has to travel (through the apparatus, conduit and patient interface). Also where a sampling line is used to measure the gas parameter (of the target gas) at the patient, there might be a delay due to the time it takes the sample to enter the sampling line. So, when referring to “at the time of the first and second flow rates”, “at the time of the first and second gas fractions”, “at the time of the first or second partial pressure” or similar, that refers to the time at which gas flow at the first and second flow rates reaches the patient (including sampling line where appropriate). If the delay of the flow rate/gas proportion change through the gas flow path is not significant, then the gas parameter is measured at the patient more or less at the same time as the flow rate/gas proportion changes. However, if there is a delay of flow rate/gas proportion change propagating through the gas path, the measurement at the patient might take place at some time (i.e. after a delay) after the flow rate/gas proportion changes is instigated at the breathing apparatus 10, to take into account the time it will take for the new flow rate/gas proportion to reach the patient. This delay can be ascertained and/or implemented in any suitable way, such as by experimentation, modelling, measuring, calculation or the like. Reference herein to “at the time of” should be read conceptually to encompass either time that the flow rate/gas proportion change reaches the patient and/or at any time later after the change of the flow rate/gas proportion at the apparatus due to the delay of the change in flow rate/gas proportion reaching the patient. This comment applies to any embodiments herein.

Where the target gas is CO₂, the invention can be used to monitor expired CO₂ and/or determine end tidal CO₂ when a patient is provided with a high flow of gases. Currently, CO₂ in displayed traces show a diluted measurement. Where the target gas is O₂, the present embodiments can monitor expired O₂ and/or determine fraction of expired O₂ (F_{E}O₂). Measuring O₂ is useful for example during a pre-oxygenation phase of a general anaesthetic procedure where a patient is pre-oxygenated to increase their O₂ levels prior to anaesthetic apnea (i.e. apnea induced by anaesthetic drugs) or during procedural sedation, during the pre-oxygenation phase before sedatives are administered and during the sedation phase where the patient is sedated and may be shallowly breathing. During the pre-oxygenation phase, O₂ from the apparatus gas flow 11 is taken up by the patient such that F_{E}O₂ in exhaled gases will rise from the start of the pre-oxygenation phase to the completion of the pre-oxygenation phase. F_{E}O₂ may provide useful information to the medical professional of O₂ levels in the patient's blood, particularly in situations where obtaining the patient's arterial blood gas measurements is not possible and/or practical. The patient's O₂ levels would preferably rise during the pre-oxygenation phase.

The embodiments described may be applied to any other situations where knowing any end tidal or exhaled gas fractions might be useful while the patient is receiving respiratory support. The invention could be used in anaesthetic procedures (i.e. operating theatres), ICUs, wards, emergency departments and the like.

### 2. General Embodiment—Varying Apparatus Gas-Flow Flow Rate

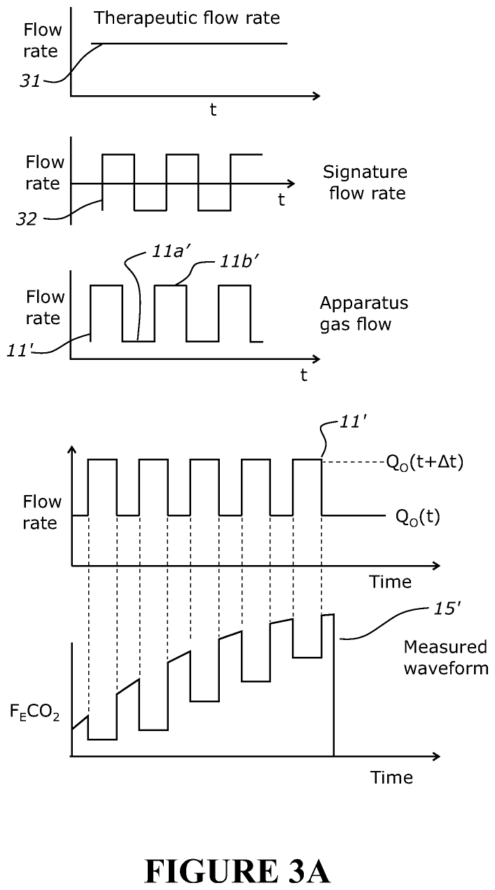

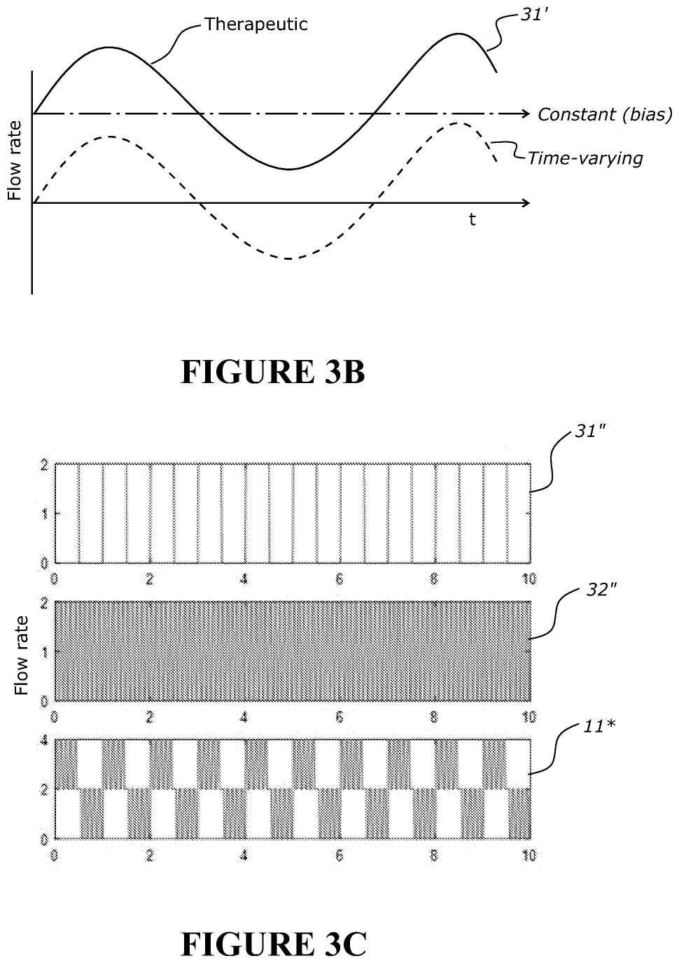

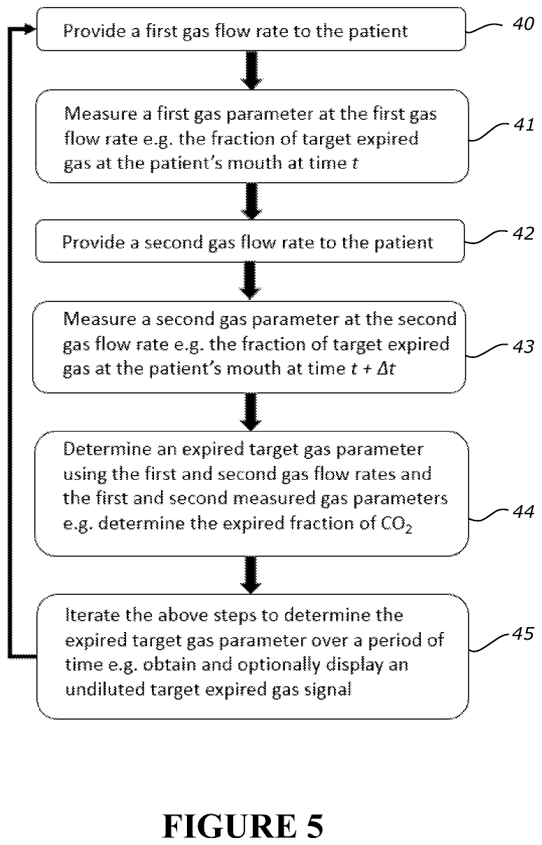

An embodiment is described, referring to the diagram and graphs of FIG. 3A and the flow diagram in FIG. 5, In general terms, determining a gas parameter is achieved by varying the flow rate (over time) of the apparatus gas flow 11′ in a known manner and using knowledge of that time-varying flow rate and information obtained from the composite gas outflow 15′ determine the parameter of the desired gas component in the actual exhaled gas flow 13′. The reference numeral 11′ is used for a varying apparatus gas flow, to distinguish from previously used reference numeral 11 that was used for an apparatus gas flow that does not vary for explanatory purposes. Likewise leak gas flow 12′, exhaled gas flow 13′, and composite outflow 15′ reference numerals are used where the apparatus gas flow is varying, instead of 11, 12, 13, 15 as used for the same parameters but where the apparatus gas flow does not vary.

As shown in FIG. 3A, a time-varying apparatus gas flow 11′ is provided to the patient by the respiratory apparatus 10. This apparatus gas flow with a time-varying flow rate now comprises at least two flow rate components. First is a therapeutic flow rate component 31 according to what is required by therapy. Second, is a signature (time-varying) flow rate component 32 that varies over time and modifies/modulates the therapeutic flow rate, over and above what is required by therapy (including any time-varying of the flow that might be required for the therapy) but in a manner that does not impact on the efficacy of the therapy provided by the apparatus gas flow. The two components 31, 32 add together to provide the overall time-varying apparatus gas flow 11′. In one alternative, the apparatus gas flow can be configured such that at times the varying flow rate goes to zero. The modified time-varying gas flow could be provided at all times, or optionally just during expiration of the patient, so as to reduce any influence on respiratory support the signature might have. Any control can be implemented in the controller or any other suitable apparatus. Note, this is an explanation of the components of the time-varying flow rate, but not necessarily an explanation of the how the time-varying flow rate is achieved. It could be achieved in numerous ways, such as described in the applicant's publications WO2015033288 or US2016/0193438, WO2016157106 or US2018/0104426, WO2017187390 or U.S. Ser. No. 16/096,660, which are incorporated herein by reference in their entirety.

As described above, the therapeutic flow rate might be a constant flow rate, but could also have time-varying flow rate component itself (that is, a varying gas flow rate with one or more time-varying flow rate components that is in addition to the signature flow). For example, as shown in FIG. 3B, the therapeutic flow rate 31′ itself comprises multiple components, including a constant (e.g. bias/base) component 31A′, and a time-varying component 31B′, which together add up to a time-varying component (herein after, any reference to varying flow rate means time-varying, even if not stated, as context allows). This can then be added to the signature flow rate (that is, the time-varying therapeutic flow rate 31′ is modified/modulated by the signature flow rate 32) to create an apparatus gas flow.

FIG. 3C is yet another example of a therapeutic flow rate 31″ with a time-varying flow rate component—this time a square wave. Also shown is a square wave signature flow rate 32″ leading to the time-varying apparatus gas flow 11*

The signature flow rate might, for example, simply have a flow rate that varies from a first flow rate to a second gas flow rate over time, but alternatively it could have any sort of varying flow rate over time, such as an oscillating flow rate or any other time varying flow rate, be it periodic (regular or irregular), non-periodic, random, non-repeating or the like. It is not necessary for there to be a regular periodic variation (e.g. a fixed frequency oscillation is not required—in fact it could be varying frequency). Also it is not necessary for amplitude to be fixed. For example, the signature flow rate may be in the form of a square wave as shown in FIG. 3A. The signature flow rate could also be a step function, sawtooth, sine wave, or a more complex random, repeating or nonrepeating function, or any other option where it varies over time between at least two different flow rates. Or it could be a combination of one or more waves, such as sine waves with various magnitudes and frequencies.

The signature flow rate component adds to (modifies/modulates) the therapeutic flow rate component to provide the varying apparatus gas flow rate 11′. Therefore, varying flow rate means any flow rate that changes at least once over time. The flow rate of the apparatus gas flow 11′ will vary and comprise a therapeutic flow rate (which may be constant, or itself might be varying, and therefore comprising various flow rate components itself) and a signature flow rate providing an additional component to vary the therapeutic flow rate of the therapeutic gas flow rate. Preferably, the frequency of the signature flow rate (where it is repeating) or the time period over which it changes (if it is not repeating) is higher than the frequency of the breathing of the patient and/or is higher than the frequency of any variation of the therapeutic flow rate component. Although, this is not essential again, this is an explanation of the components of the time-varying signature flow rate component, but not necessarily an explanation of the how the time-varying flow rate component is achieved. Any suitable apparatus for varying of a gas source to obtain a time varying flow rate with the characteristics above could be implemented. For example, a time varying flow rate could be achieved in numerous ways, such as described in the applicant's publications WO2015033288 or US2016/0193438 (e.g. FIGS. 56 to 57), WO2016157106 or US2018/0104426, WO2017187390 or U.S. Ser. No. 16/096,660 all of which are incorporated herein by reference in their entirety. Particular non limiting examples could be a controllable valve, and/or a speed controllable motor/impeller arrangement.

As an example, FIG. 3A shows a varying flow rate of an apparatus gas flow 11′ which comprises the gas flow rate generated for therapeutic purposes 31, and the time-varying (signature) component 32. The time-varying signature component is a square wave function, which provides a regular repeating periodic varying flow rate. When the leak gas flow rate 12′ adds to the exhaled gas flow of the patient 13′, this creates a composite gas outflow (total flow) 15′ that has the signature flow rate 32 as a component see e.g. FIG. 3A, item 32. When measuring the gas parameter in the composite gas outflow (see example of measuring CO₂ fraction at the bottom of FIG. 3A), the time-varying flow rate 32 of the apparatus gas flow 11′ affects the gas parameter in the composite gas outflow 15′, and becomes apparent in the measurement of the gas parameter.

The flow rate of the apparatus gas flow 11′ in combination with measurement of the parameter of gas component of the composite gas outflow 15′ over time of the gas outflow parameter can be utilised to:
a) determine the effect the apparatus gas flow has on the parameter of the exhaled apparatus gas flow 13′, and/or
b) determine the gas parameter of the actual exhaled patient gas flow 13′.

This determining of a) and/or b) is achieved by any suitable means, such as filtering, interpolating or extrapolating the composite gas outflow 15′ to obtain the gas flow parameter of the exhaled flow, modelling the gas flow parameter from the composite gas outflow 15′, calculating or otherwise determining the gas flow parameter from the composite gas outflow. Providing a signature flow rate 32 to vary the apparatus gas-flow flow rate 11′ changes the gas fraction (or other parameter being measured) in the composite gas outflow 15′ and allows the effect of the change in gas fraction to be removed from the underlying expired gas signal (exhaled gas flow 13), in a suitable manner, either directly or indirectly. The change in gas fraction could be a dilution or an increased fraction of the gas. Interpolation can be used to recover the waveform and value of the expired gas, as one example. As another example, a measure of the patient gas-flow flow rate at two time points and the parameter of the gas component in the composite gas outflow at the same two time points can be used to determine the parameter of the gas component in the exhaled flow. As an example, the proportion (e.g. fraction) of CO₂ in the exhaled gas flow can be determined by measuring/knowing the apparatus gas flow (flow rate) at two times, and the CO₂ proportion (e.g. fraction) in the composite gas outflow at the same times. This can be repeatedly carried out at other times as the apparatus gas flow flow rate varies over time. Other examples are possible too. As another example, in FIG. 3A, the actual gas flow parameter can be extrapolated from the measurement, as shown. In one alternative, the apparatus gas flow flow rate can be configured such that at times the varying flow rate goes to zero, which makes the determination easier.

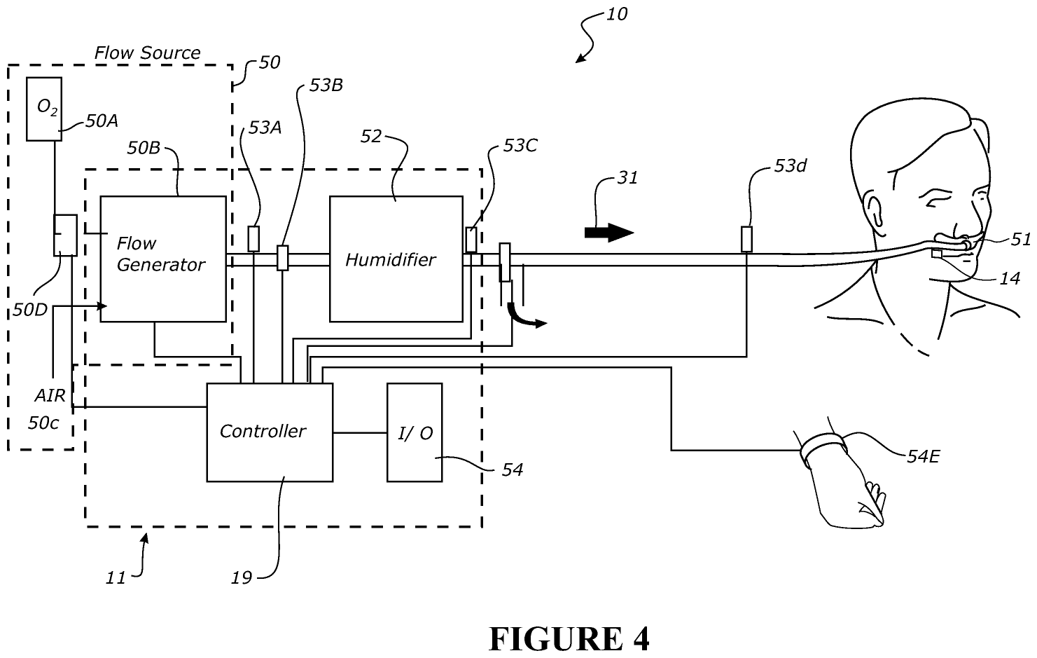

An apparatus and method for achieving determining the gas parameter as described above will be described with reference to FIGS. 4 and 5. This apparatus can also be used for other embodiments described herein.

FIG. 4 shows a respiratory apparatus 10 for providing flow therapy or other therapy to a patient. The apparatus is configured for delivering a time-varying apparatus gas flow 11′ and determining the parameter of the desired gas component of the exhaled gas flow 13′. The apparatus 10 could be an integrated or a separate component based arrangement, generally shown in the dotted box in FIG. 4. In some configurations, the apparatus could be a modular arrangement of components. As such, the apparatus could be referred to as a “system” but the terms can be used interchangeably without limitation. Hereinafter it will be referred to as an apparatus, but this should not be considered limiting. The apparatus may be used for any suitable purpose including preoxygenation during an anaesthetic procedure, during an anaesthetic procedure, high flow therapy, ventilation, whilst treating patients in respiratory distress, treating patients with obstructive sleep apnoea or anywhere else where monitoring of an aspect of patient breathing is required.

The apparatus comprises a flow source 50 for providing a high flow gas 31 such as oxygen, or a mix of oxygen and one or more other gases. Alternatively, the apparatus can have a connection for coupling to a flow source. As such, the flow source might be considered to form part of the apparatus or be separate to it, depending on context, or even part of the flow source forms part of the apparatus, and part of the flow source fall outside the apparatus.

The flow source could be an in-wall supply of oxygen, a tank of oxygen 50A, a tank of other gas and/or a high flow therapy apparatus with a blower/flow generator 50B. FIG. 4 shows a flow source 50 with a flow generator 50B, with an optional air inlet 50C and optional connection to an O₂ source (such as tank or O₂ generator) 50A via a shut off valve and/or regulator and/or other gas flow control 50D, but this is just one option. The description from here can refer to either embodiment. The flow source could be one or a combination of a flow generator, O₂ source, air source as described. The flow source 50 is shown as part of the apparatus 10, although in the case of an external oxygen tank or in-wall source, it may be considered a separate component, in which case the apparatus has a connection port to connect to such flow source. The flow source provides a (preferably high) flow of gas that can be delivered to a patient via a delivery conduit, and patient interface 51. Depending on the end-use, the patient interface 51 may be an unsealed (also termed “non-sealing”) interface (for example when used in high flow therapy) such as a nasal interface (cannula), or a sealed interface (for example when used in CPAP) such as a nasal mask, full face mask, or nasal pillows. The time-varying flow rate embodiment can be used with a non-sealing patient interface. A time-varying flow rate gas flow is not passed to or through a cavity external to the patient, so e.g. is preferably passed through a non-sealing nasal cannula. An external cavity might introduce a low pass filter that might attenuate a time-varying flow rate signature. The time-varying fraction embodiment can be used with a sealed patient interface also. The patient interface 51 is preferably a non-sealing patient interface which would for example help to prevent barotrauma (e.g. tissue damage to the lungs or other organs of the respiratory apparatus due to difference in pressure relative to the atmosphere). The patient interface may be a nasal interface (cannula) with a manifold and nasal prongs, and/or a face mask, and/or a nasal pillows mask, and/or a nasal mask, and/or a tracheostomy interface, or any other suitable type of patient interface. The flow source could provide a therapeutic gas flow rate of between, e.g. about 0.5 litres/min and about 375 litres/min, or any range within that range, or even ranges with higher or lower limits.

The time-varying apparatus gas flow may have an therapeutic time-varying (e.g. oscillating) flow rate and the controller controls a gas flow modulator to provide the therapeutic time-varying apparatus gas flow with an oscillating flow rate of: about 375 litres/min to about 0 litres/min, or preferably of about 240 litres/min to about 7.5 litres/min, or more preferably of about 120 litres/min to about 15 litres/min, and/or the oscillating flow rate has one or more frequencies of about 0.1 Hz to about 200 Hz, and preferably about 0.1 Hz to about 6 Hz, and more preferably about 0.5 Hz to about 4 Hz, and more preferably 0.6 Hz to 3 Hz. The gas flow modulator might be the flow source (where that could be a flow generator, O2 source, ambient air or the like as previously discussed) and/or a valve or other device to modulate or otherwise vary parameters (e.g. flow rate, gas proportion) of a gas flow gas flow.

The oscillating flow rate may comprise a therapeutic flow rate component, wherein the therapeutic flow rate is about 375 litres/min to about 0 litres/min, or about 150 litres/min to about 0 litres/min, or is preferably about 120 litres/min to about 15 litres/min, or is more preferably about 90 litres/min to about 30 litres/min.

The oscillating flow rate may comprise a therapeutic gas flow component, wherein the constant (e.g. bias/base) flow rate component of the therapeutic gas flow is about 0.5 litres/min to about 25 litres/min.

The oscillating flow rate may comprise a therapeutic flow rate component, wherein the therapeutic flow rate is about 0.2 litres/min per patient kilogram to about 2.5 litres/min per patient kilogram; and preferably is about 0.25 litres/min per patient kilogram to about 1.75 litres/min per patient kilogram; and more preferably is about 0.3 litres/min per patient kilogram to about 1.25 litres/min or about 1.5 litres/min per patient kilogram; and more preferably is about 0.4 litres/min per patient kilogram to about 0.8 litres/min per patient kilogram.

The one or more components of the time-varying (e.g. oscillating) gas flow may have one or more frequencies of about 0.3 Hz to about 4 Hz.

The oscillating flow rate may comprise at least one time-varying flow rate component, wherein each oscillating flow rate is about 0.05 litres/min per patient kilogram to 2 litres/min per patient kilogram; and preferably is about 0.05 litres/min per patient kilogram to about 0.5 litres/min per patient kilogram; and preferably about 0.12 litres/min per patient kilogram to about 0.4 litres/min per patient kilogram; and more preferably about 0.12 litres/min per patient kilogram to about 0.35 litres/min per patient kilogram. Alternatively, the oscillating flow rate may comprise at least one time-varying flow rate component, wherein each oscillating flow rate is in the range of 0.05 litres/min per patient kilogram to 2 litres/min per patient kilogram; and preferably in the range of 0.1 litres/min per patient kilogram to 1 litres/min per patient kilogram; and more preferably in the range of 0.2 litres/min per patient kilogram to 0.8 litres/min per patient kilogram.

The above are examples of therapeutic time-varying flow rates. Signature flow rates could be provided also and could be lower, the same or higher than therapeutic flow rates. Signature flow rate frequencies could be lower, the same or higher than therapeutic flow rate frequencies (when time-varying). In some embodiments, the signature flow rate has a higher frequency that the therapeutic flow rate.

As an example, the signature flow rates can step between a first flow rate and second flow rate, one or both of which can fall in the range of about 0 LPM to 70 LPM. The maximum signature flow rate could be the therapeutic flow rate. The signature flow rate could be combined with the therapeutic flow rate (e.g. added) or it could form part or all of the therapeutic flow rate—that is, the therapeutic flow rate itself could be the signature flow rate. In some embodiments, the signature flow rate can be related to the therapeutic flow rate as a percentage. For example, the signature time-varying flow rate (adults) is in the range of:
about 0% to about 200% of the therapeutic flow rate,
about 0% to 100% of the therapeutic flow rate,
about 100% to 200% of the therapeutic flow rate, or
about 50% to 150% of the therapeutic flow rate, and/or is in the range of
about 0-140 LPM,
about 0-70 LPM,
about 70-140 LPM,
about 40-100 LPM, or
about 20-60 LPM

These are not limiting flow rates, and it should also be noted that the signature flow rates could be negative, but when combined with the therapeutic flow rates create a total flow rate that is positive.

In some embodiments, the therapeutic flow rates might also serve as signature flow rates. That is, they perform a dual purpose.

The above are just example, and other types of time-varying flow rates could be provided and the controller controls a gas flow modulator to provide the time varying apparatus gas flow with the time-varying flow rate. The apparatus can have knowledge of the time-varying flow rate, and/or can measure the time-varying flow rate provided, e.g. by a flow sensor, e.g. 53A, 53B.

A humidifier 52 can optionally be provided between the flow source 50 and the patient to provide humidification of the delivered gas. This could be a humidifier integrated with the flow source 10 to form an integrated apparatus 59 (see dotted lines) or separate but attachable to the flow source 10. Alternatively, the humidifier 52 could be a standalone humidifier with a chamber and base, where the humidifier is coupled to the flow source 10 via conduits or other suitable means. One or more sensors 53A, 53B, 53C, 53D such as flow rate, oxygen fraction or other gas fraction, full or partial pressure, humidity, temperature or other sensors can be placed throughout the apparatus and/or at, on or near the patient 16. Alternatively, or additionally, sensors from which such parameters can be derived could be used. In addition, or alternatively, the sensors 53A-53D can be one or more physiological sensors for sensing patient physiological parameters such as, heart rate, oxygen saturation (e.g. pulse oximeter sensor 54E), partial pressure of oxygen in the blood, respiratory rate, partial pressure of O₂ and/or CO₂ in the blood. Alternatively, or additionally, sensors from which such parameters can be derived could be used. Other patient sensors could comprise EEG sensors, torso bands to detect breathing, and any other suitable sensors. In some configurations the humidifier may be optional, or it may be preferred due to the advantages of humidified gases helping to maintain the condition of the airways. Humidification is preferably used with high flow gas flows to increase patient comfort, compliance, support and and/or safety. One or more of the sensors might form part of the apparatus, or be external thereto, with the apparatus having inputs for any external sensors.

A sensor 14 for measuring the gas parameter (of the target gas) of the patient composite gas outflow 15 is provided. That is, depending on the target gas e.g. oxygen, carbon dioxide, nitrogen, helium and/or an anaesthetic agent such as sevoflurane, a sensor is chosen to sense that gas in the composite gas outflow. The sensor can be a mainstream or side stream sensor, and can be placed proximate (in, on, near) the nose and/or mouth. Other positions are possible. The time-varying flow rate embodiment can work with one gas parameter sensor—e.g. it can work when one gas parameter (e.g. fraction CO₂, or fraction O₂) is measured. In the time-varying flow rate embodiment, it is not essential to measure more than one gas parameter to obtain the target parameter (e.g. it is not necessary to measure fraction CO₂ and fraction O₂ to implement the embodiment herein).

The output from the sensors is sent to a controller to assist control of the apparatus, including among other things, to vary gas flow. Alternatively, or additionally, input could come from a user. The controller is coupled to the flow source, humidifier and sensors. It controls these and other aspects of the apparatus to be described below. The controller can operate the flow source to provide the delivered flow of gas. It can also operate the gas flow modulator(s) (including the flow source) to control the flow, pressure, volume and/or other parameters of gas provided by the flow source based on feedback from sensors, or optionally without feedback (e.g. using default settings). The controller can also control any other suitable parameters of the flow source to meet oxygenation requirements and/or CO₂ removal. The controller 19 can also control the humidifier 52 based on feed-back from the sensors 53A-53D, 14. Using input from the sensors, the controller can determine oxygenation requirements and provide information to a medical professional (who may control the components of the respiratory apparatus to provide the desired therapy, e.g. flow rate, O₂ fraction, humidity, etc.) and/or control parameters of the flow source, gas flow modulator(s) and/or humidifier as required. Alternatively, the embodiments could be provided as a standalone monitoring apparatus, independent of a respiratory apparatus that provides information to a medical professional and/or communicate and control components of the respiratory apparatus to provide a desired therapy. The medical professional can then control the respiratory apparatus to provide the desired therapy. Accordingly, the controller may not always determine oxygenation requirements and control parameters of the apparatus.

The controller 19 is also configured to operate the apparatus so that the apparatus gas flow has a time-varying flow rate that provides therapy and also a signature flow rates as described. It can do this through any suitable means such as controlling flow generator 50B or any other suitable gas modulator. The gas modulator can be used to modulate (that is, varying, modify, adjust or otherwise control parameters of the gas flow). Each gas flow modulator can be provided in the flow source (and the flow source itself can be a gas flow modulator), after the flow source and before the humidifier, after the humidifier, and/or in any other suitable place in the apparatus to modulate gas flow path. It can also operate the gas flow modulator(s) (including the flow source) to control the flow, pressure, volume and/or other parameters of gas provided by the flow source based on feedback from sensors, or optionally without feedback (e.g. using default settings). The controller can also control any other suitable parameters of the flow source to meet oxygenation requirements. The gas modulator could be, for example, anything described in WO2017/187390 or U.S. Ser. No. 16/096,660 which are incorporated herein by reference in their entirety.

The controller can then measure the composite gas outflow and determine the gas parameter using any of the following techniques.

For other embodiments below relating to varying gas proportion, the controller 19 is additionally or alternatively be configured to operate the apparatus so that the apparatus gas flow has a time-varying gas proportion (such as O₂ fraction or other gas fraction and/or O₂ partial pressure or other gas partial pressure) that provides therapy/respiratory support, and also a signature gas proportion (such as gas fraction and/or gas partial pressure) as described. It can do this through any suitable means such as controlling a proportional valve coupled to an O₂ source 50A or any other means previously described in other patents. The controller can then measure the composite gas outflow and or determine (e.g. obtain an estimate of) the gas parameter using any of the following techniques. In one embodiment, there are two proportional valves that operate 180 degrees out of phase. As one opens, the other closes. One controls O₂ fraction in the apparatus gas flow, and the other controls Air fraction in the gas flow, but together keeping the total gas flow rate constant. In another alternative, a single proportional valve is used with an impeller where the proportional valve controls an O₂ fraction and the impeller controls the flow rate. In some embodiments, the single proportional valve may be used before or after the impeller. Where the single proportional valve is used before the impeller, the proportional valve controls the O₂ fraction into the inlet of the impeller along with the ambient air. In some embodiments, more than one proportional valve may be used with an impeller and may be positioned anywhere in the system with respect to the impeller. The controller 19 can control the proportional valve(s) to operate as required to achieve the time-varying gas proportion as described herein.

An input/output interface 54 (such as a display and/or input device) is provided. The input device is for receiving information from a user (e.g. clinician or patient) that can be used for example for determining oxygenation requirements, anaesthetic gas agent, detection, flow rates, gas fractions, partial pressures and/or any other parameter that might be controlled by the apparatus.

The apparatus can also be operated to determine dose/oxygenation requirements (hereinafter “oxygen requirements”) of a patient for/in relation to general anaesthesia (that is, the oxygen requirements pre-anaesthesia during a pre-oxygenation phase and/or the oxygen requirements during anaesthesia—which might include when the patient is apnoeic or when the patient is breathing), as well as after such a procedure, which may include the extubation period. The apparatus 10 is also configured to adjust and provide high flow gas to a patient for the purposes of anaesthetic procedures and adjust the parameters of the high flow gas (such as pressure, flow rate, volume of gas, gas composition) delivered to the patient as required to meet oxygenation requirements. The apparatus also comprises a display which can be part of the I/O for displaying the measure of the gas parameter of the exhaled gas flow, as a graph, digital readout or any other suitable means.

FIG. 5, which shows a method of operating a respiratory apparatus 10 with a sensor(s) 14 and controller 19 as per described above. The following steps can be undertaken. A time varying apparatus gas flow 11′ comprising a time-varying flow rate with a signature is generated and provided to the patient. In one variation, the time-varying flow rate comprises at least a therapeutic flow rate component 31. The therapeutic gas flow rate is modified/modulated by (e.g. in this case by adding) a signature gas flow rate component 32 that varies over time to create an apparatus gas flow 11′ with a time-varying flow rate with at least a second flow rate (at a later time e.g. 11b′) that is different to the first flow rate (at the earlier time e.g. 11a′). This could be a simple step change, or as noted above a more complex time-varying flow rate waveform, such as a square wave, sinusoidal wave or any other wave described herein or envisaged. The “modified” apparatus gas flow 11′ is provided to the patient. The first flow rate 11a′ of the apparatus gas flow 11′ is provided to the patient, step 40, and the second flow rate 11b′ of the apparatus gas flow 11′ is provided to the patient sometime later, step 42. The flow rates are preferably known, or alternatively measured. At the same time, the respiratory apparatus 10 is monitoring the composite gas outflow 15′ over time, which is a combination of the leak gas flow 12′ (with a time-varying flow rate) and the exhaled gas flow 13′. This comprises at least measuring a first parameter at the first flow rate 11a′ (in this case CO₂ fraction) of the composite gas outflow 15′ sometime later correlating to the first flow rate 11a′ being provided, step 41, and the same gas parameter (CO₂ fraction) of the composite gas outflow at the second gas flow rate 11b′ at a later point in time, step 43.

The apparatus 10 could compensate for a delay in providing the first flow rate and the measurement of the first parameter 11a′ in the composite gas outflow 15′ affected by the first flow rate, for example in the case of side stream sampling, a correction of “X” seconds could be applied.

The parameter of the composite gas outflow 15″ is measured at or near the mouth and/or nose (“proximate”) of the patient using the sensor 14. In some embodiments, only one gas parameter (the target gas parameter of interest) need be measured. The gas parameter in the exhaled gas flow 13′ is then determined using the (preferably known, but optionally measured) first and second gas flow rates and the measured gas parameter at the first and second points in time, step 44. In some embodiments, the first or second flow rate is 0 L per minute. In some embodiments for adults, the first and second flow rates are greater than or equal to about 0 L per minute, and preferably about or greater than about 20 L per minute, and more preferably between about 20 L per minute to about 90 L per minute. In some embodiments for premature/infants/paediatrics (with body mass in the range of about 1 to about 30 kg) the therapeutic flow can be set to 0.4-0.8 L/min/kg with a minimum of about 0.5 L/min and a maximum of about 25 L/min. For patients under 2 kg maximum flow is set to 8 L/min. The oscillating flow is set to 0.05-2 L/min/kg with a preferred range of 0.1-1 L/min/kg and another preferred range of 0.2-0.8 L/min/kg.

Steps 40 to 44 can be repeated continuously/periodically, step 45, to determine the gas parameter of the exhaled gas flow 13′ over time as the apparatus gas flow 11′ varies its flow rate 11a′, 11b′ over time due to the varying signature 32 flow rate. The parameter of the gas component can be measured in the composite gas outflow 15′ during expiration phase of the patient breathing. While simply a first 11a′ and second 11b′ gas flow rate in the apparatus gas flow 11′ could be used, in practice the signature gas flow 32 is likely to be varying continuously, or at least periodically/discretely over time and the composite gas outflow 15′ can be measured continuously or periodically (e.g. at a sampling rate) to get continuous or periodic measures of the exhaled gas flow parameter, which can then be displayed via a graph or display to give a real-time measure of the exhaled gas flow parameter.

Various hardware configurations and methods of operation can implement the invention as described in overview above. Examples of those, without limitation, are set out below.

### 3. Exemplary Embodiment—Varying Apparatus Gas-Flow Flow Rate

One exemplary embodiment, will now be described with reference to the FIGS. 1 to 5, using the apparatus, and control methods described above. Any control method can be implemented in the controller or any other suitable apparatus. In this embodiment, the medical professional desires to monitor exhaled CO₂ or O₂ or an anaesthetic agent such as Sevoflurane. Measurement of expired gas fraction can be used by a medical professional to perform a variety of monitoring functions including checking that gas exchange is occurring in patients. For example, the expired fraction of oxygen, F_{E}O₂ might be measured to assess the effectiveness of pre-oxygenation before anaesthesia. The expired fraction of carbon dioxide, F_{E}CO₂, is an indicator of gas exchange. The monitoring of F_{E}CO₂ is recommended or mandated in a number of anaesthetic standards. However, as noted above, monitoring FeCO2 and/or FeO2 can be challenging when a gases flow is provided to a patient, especially if the gases flow is provided at high flow rates, which will be explained below. The embodiment addresses that challenge. The embodiment utilises a non-sealing respiratory apparatus, e.g. with a non-sealing nasal cannula.

Nasal high flow (NHF) is used to provide respiratory support, typically through a non-sealing nasal interface (cannula), as shown in FIG. 4. The apparatus may have a CO₂ sampler (sensor 14) such as that described in WO2018070885 or U.S. Ser. No. 16/341,767 which are incorporated herein by reference in their entirety. The nasal cannula comprises prongs that are configured to insert into the nares of the patient in use. The prongs are sized to provide a gap between the prongs and the walls of the patient's nares such that the prongs do not seal with the patient's nares. This allows for example the entrainment of ambient gases into the patient's airways in certain situations and/or exhaled gases to flow around the prongs and out to ambient.

The sampler is an attachment to the nasal high flow interface (cannula) that allows measurement expired gases such as carbon dioxide. In use, expired gas is transported (actively or passively) through the sampling line and into the measurement device. This is side stream sampling although the embodiments described here may also be implemented with mainstream sampling, where a CO₂ sensor is located in the main flow path. The sampler may also be adapted to transport and/or sample other exhaled gases such as O₂. The portion of the sampler that captures a portion of the expired gas for sampling may be manoeuvrable between the patient's nares and mouth.

Leak gas flow 12′ from the apparatus 10 changes the gas fraction (by diluting or increasing the gas fraction) in the composite gas outflow 15′. As previously noted, reliable measurement of the expired fraction of carbon dioxide and/or oxygen in an exhaled gas flow 13′ during high flow is difficult. For example, in the case of pre-oxygenation, where the fraction of inspired oxygen is typically 1, the oxygen from the high flow apparatus provides an artificially high value and limits the clinical usefulness of the measurement. The expired fraction of oxygen provides insight into effectiveness of pre-oxygenation for example whether pre-oxygenation is sufficient enough whereby the fraction of oxygen in the patient's lungs is at sufficient level to provide a desired safe apnoea time in a procedure.

Note the following related to safe apnoea time from WO2018/185714 or U.S. Ser. No. 16/500,329 which are incorporated herein in their entirety. The duration of safe apnoea is defined as the time until a patient reaches a specified oxygen saturation level. Typically, that oxygen saturation level may be 88-90%, preferably 90-92%, although that level may vary depending on the patient and procedure being carried out. Saturations below this level can rapidly deteriorate to critical levels (<70%, preferably <80%) on the steep section of the oxyhaemoglobin dissociation curve posing significant risk to the patient. Alternatively, the duration of safe apnoea is defined as the time until a patient reaches a specified level of arterial CO2.

The purpose of the embodiment is to accurately measure and display the waveform and magnitude of an expired gas in the presence of nasal high flow (NHF). This is done by the use of an oscillating flow or a flow that steps between at least two different flow rates.

The apparatus is operated to provide an apparatus gas flow 11′ with a time-varying flow rate as previously described with a therapeutic flow rate component 31 and a signature (time-varying) flow rate component 32. Preferably this is high flow (with therapeutic component) as defined, and more preferably between 20 to 90 litres per minute. In this example, a constant therapeutic flow rate 31 is provided and is added to a square wave signature flow rate component 32 to produce the apparatus gas flow 11′ with time-varying flow rate, as shown in FIG. 3A. In some embodiments, the signature flow rate component 32 may be added to a constant therapeutic flow rate component 31 to produce the apparatus gas flow 11′ with a time-varying flow rate, as shown in FIG. 3A. The controller 19 or a user operates the flow source 10 to provide an apparatus gas flow 11′ with a flow rate. The controller 19 may be programmed with the desired apparatus gas flow 11′ with a varying flow rate and creates the varying flow rate to provide the required variation in the flow rate over time. Other methods of varying the flow rate are possible also, such as described in WO2018070885 or U.S. Ser. No. 16/341,767 which are incorporated herein by reference in their entirety.

In use, the patient breathes in the apparatus gas flow 11′ and exhales a gas flow 13′. The exhaled gas flow 13′ (with a CO₂ fraction) combines with the leak gas flow 12′ to produce a composite gas outflow 15′ as shown in FIG. 3A (with a diluted CO₂ fraction)

The sensor 14 measures the diluted CO₂ (or O₂ in other variations) fraction in the composite gas outflow and passes this information to the controller. The output of the sensor measuring the parameter of the composite gas outflow 15′ is represented as the waveform 15′ shown in FIG. 3A. The controller then needs to process that output to determine the actual CO₂ (or O₂) fraction in the exhaled patient gas flow.

The fraction of exhaled gas component, e.g. CO₂ (that is, F_{E}CO₂)(or O₂—that is F_{E}O₂), as a percentage of the total gas exhaled per volume can be determined from the composite gas outflow using equations that utilise knowledge of the change in flow rate of the time-varying apparatus gas flow. This determination can be obtained based on the premise that the fraction of exhaled gas (component) can be determined from the known/measured quantities:

Fₘ(t), volume fraction of the gas component measured in the patient composite gas outflow 15′ from the patient (this is the measured CO₂/O₂ fraction parameter of the composite gas outflow 15′, preferably measured by the sensor 14) at time t.

Fₘ(t) is preferably measured at the mouth of the patient when the patient's mouth is open or the nose if the patient's mouth is closed.

Qₒ(t), flow rate of the apparatus gas flow 11′ provided from the respiratory apparatus to the patient (apparatus gas flow rate) at time t.

Fₘ(t+Δt), volume fraction of the gas component measured in the patient composite gas outflow 15′ (this is the measured CO₂/O₂ fraction parameter of the composite gas outflow, preferably measured by the sensor 14) at time t+Δt. Fₘ(t+Δt) is preferably measured at the mouth of the patient when the patient's mouth is open or the nose if the patient's mouth is closed.

Qₒ(t+Δt), flow rate of apparatus gas flow 11′ provided from the respiratory apparatus to the patient (apparatus gas flow rate), at time t+Δt.

Fₒ(t), volume fraction of the gas component in the apparatus gas flow 11′ coming from the respiratory apparatus, at time t and t+Δt.

Fₒ(t+Δt), volume fraction of the gas component in the apparatus gas flow 11′ coming from the respiratory apparatus, at time t+Δt.

Using the equation (4)

$${F}_{E} ( t ) = \frac{{Q}_{o} ( t ) {F}_{m} ( t + Δ t ) ( {F}_{o} ( t ) - {F}_{m} ( t ) ) - {Q}_{o} ( t + Δ t ) {F}_{m} ( t ) ( {F}_{o} ( t + Δ t ) - {F}_{m} ( t + Δ t ) )}{{Q}_{o} ( t ) ( {F}_{o} ( t ) - {F}_{m} ( t ) ) - {Q}_{o} ( t + Δ t ) ( ( {F}_{o} ( t + Δ t ) - {F}_{m} ( t + Δ t ) )} \qquad ( 4 )$$

Where

Qₒ is the flow rate of the apparatus gas flow 11′

F_{O} is the volume fraction of the gas component in the apparatus gas flow 11′ coming from the respiratory apparatus

F_{E} is the volume fraction of the gas component in the exhaled gas flow (volume fraction of expired gas) 13′

Fₘ is the volume fraction of the gas component in the patient composite gas outflow 15′ from the patient

The CO₂ (or O₂) fraction in the composite gas outflow 15′ is measured by the sensor 14, and the flow rate of the apparatus gas outflow 11′ is known (or can be measured). So, by measuring the CO₂ (or O₂) fraction in the composite gas outflow 15′ using sensor 14 at two (or more) different times, and knowing/measuring the CO₂ (or O₂) fraction and flow rate of the apparatus gas outflow 11′ at the two (or more) different times, the CO₂(or O₂) fraction in the exhaled gas flow 13′ can be determined using equation (4).

In the case of measuring CO₂ or any other gas component that is not being delivered to the patient by the high flow apparatus and is expired by the patient, equation 4 can be simplified. For measurements of CO₂, Fₒ(t)=Fₒ(t+Δt)˜0 and so equation (4) reduces to:

$${F}_{E} ( t ) ∼ \frac{{Q}_{o} ( t + Δ t ) {F}_{m} ( t ) {F}_{m} ( t + Δ t ) ) - {Q}_{o} ( t ) {F}_{m} ( t + Δ t ) {F}_{m} ( t )}{{Q}_{o} ( t + Δ t ) {F}_{m} ( t + Δ t ) - {Q}_{o} ( t ) {F}_{m} ( t )} \qquad ( 5 )$$

$${F}_{E} ( t ) ∼ {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{o} ( t ) {F}_{m} ( t )}$$

Equation (5) provides F_{E} as a function of:

Qₒ(t),Qₒ(t+Δt),Fₘ(t+Δt),Fₘ(t)

In practice, the CO₂ (or O₂) fraction will be measured continuously or periodically to go with a real-time output, which can then be used in combination with knowledge/measurement of the apparatus gas flow 11′ that is also sampled continuously or periodically to determine a real-time CO₂ (or O₂) fraction of the exhaled gas flow. This can be output as a graph, digital readout or the like on the display. In practice, the flow rate will vary continuously, or discretely but periodically or regularly over a time period to provide a plurality of data points. Therefore, by varying the flow rate at least once, the difference between a first 11a′ and second 11b′ flow rate can be used. The controller will therefore measure the gas parameter at suitable time points and then for each measurement calculate F_{E}.

The found F_{E}CO₂ can recover the carbon dioxide waveform seen in FIG. 2.

### 4. Mathematical Derivation of Equations (4), (5)

The equations (4), (5) are derived as follows.

Assuming that all or most of the gas expired by the patient exits the mouth, the volume fraction (Fₘ) of a gas measured at the mouth of a patient being provided with nasal high flow as a function of time may be expressed as:

$${F}_{m} ( t ) = \frac{{F}_{E} ( t ) {Q}_{E} ( t ) + k ( t ) {F}_{o} ( t ) {Q}_{o} ( t )}{{Q}_{E} ( t ) + k ( t ) {Q}_{o} ( t )} \qquad ( 1 )$$

where:

Qₒ is the flow rate of the apparatus gas flow 11′

Fₒ is the volume fraction of the gas component in the apparatus gas flow 11′ coming from the respiratory apparatus'

k is the proportion of the apparatus gas flow 11′ that comes out through the patient's mouth (and (1−k) is the proportion that goes through the nose)

F_{E} is the volume fraction of the gas component in the exhaled patient gas flow

Q_{E} is the flow rate of the patient exhaled gas flow

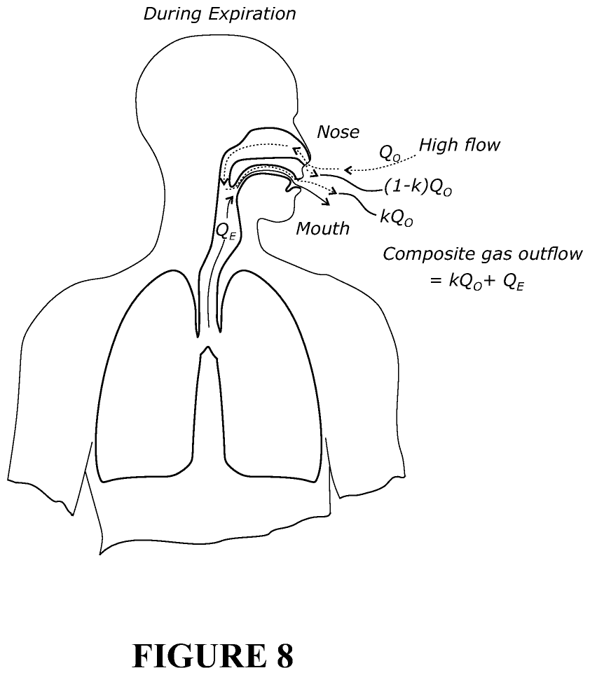

The flows are show in FIG. 8, with reference to a patient.

Equation (1) may be rearranged to find the ratio of the unknown quantities, k and Q_{E}:

$${F}_{m} ( t ) \cdot ( {Q}_{E} ( t ) + k ( t ) {Q}_{o} ( t ) ) = {F}_{E} ( t ) {Q}_{E} ( t ) + k ( t ) {F}_{o} ( t ) {Q}_{o} ( t ) \qquad ( 2 )$$

$${Q}_{E} ( t ) \cdot ( {F}_{m} ( t ) - {F}_{E} ( t ) ) = k ( t ) ( {F}_{o} ( t ) {Q}_{o} ( t ) - {Q}_{o} ( t ) {F}_{m} ( t ) )$$

$$\frac{{Q}_{E} ( t )}{k ( t )} = \frac{{F}_{o} ( t ) {Q}_{o} ( t ) - {Q}_{o} ( t ) {F}_{m} ( t )}{{F}_{m} ( t ) - {F}_{E} ( t )}$$

$$\frac{{Q}_{E} ( t )}{k ( t )} = {Q}_{o} ( t ) \frac{{F}_{o} ( t ) - {F}_{m} ( t )}{{F}_{m} ( t ) - {F}_{E} ( t )} \qquad ( 3 )$$

For two samples taken at times t and t+Δt where Δt, the time between samples, is sufficiently short such that we can assume that during the patient's expiratory phase, the fraction of the (expired) gas component (volume fraction of the gas component measured in the patient composite gas outflow 15′ Fₘ), the patient exhaled gas flow flow rate (Q_{E}) and the proportion of the apparatus gas flow exiting the mouth (k) to be approximately constant then we can approximate:

$$\frac{{Q}_{E} ( t + Δ t )}{k ( t + Δ t )} ∼ \frac{{Q}_{E} ( t )}{k ( t )}$$

$${F}_{E} ( t + Δ t ) ∼ {F}_{E} ( t )$$

Then from (3):

$${Q}_{o} ( t + Δ t ) \frac{{F}_{o} ( t + Δ t ) - {F}_{m} ( t + Δ t )}{{F}_{m} ( t + Δ t ) - {F}_{E} ( t )} \approx {Q}_{o} ( t ) \frac{{F}_{o} ( t ) - {F}_{m} ( t )}{{F}_{m} ( t ) - {F}_{E} ( t )}$$

Solving for F_{E}:

$$\frac{{Q}_{o} ( t + Δ t )}{{Q}_{o} ( t )} \cdot \frac{{F}_{o} ( t + Δ t ) - {F}_{m} ( t + Δ t )}{{F}_{o} ( t ) - {F}_{m} ( t )} \approx \frac{{F}_{m} ( t + Δ t ) - {F}_{E} ( t )}{{F}_{m} ( t ) - {F}_{E} ( t )}$$

$$( {F}_{m} ( t ) - {F}_{E} ( t ) ) \cdot \frac{{Q}_{o} ( t + Δ t )}{{Q}_{o} ( t )} \cdot \frac{{F}_{o} ( t + Δ t ) - {F}_{m} ( t + Δ t )}{{F}_{o} ( t ) - {F}_{m} ( t )} \approx {F}_{m} ( t + Δ t ) - {F}_{E} ( t ) \qquad ( 4 )$$

$${F}_{E} ( t ) \cdot \{ 1 - \frac{{Q}_{o} ( t + Δ t )}{{Q}_{o} ( t )} \cdot \frac{{F}_{o} ( t + Δ t ) - {F}_{m} ( t + Δ t )}{{F}_{o} ( t ) - {F}_{m} ( t )} \} \approx {F}_{m} ( t + Δ t ) - {F}_{m} ( t ) \frac{{Q}_{o} ( t + Δ t )}{{Q}_{o} ( t )} \cdot \frac{{F}_{o} ( t + Δ t ) - {F}_{m} ( t + Δ t )}{{F}_{o} ( t ) - {F}_{m} ( t )}$$

$${F}_{E} ( t ) \approx \frac{{F}_{m} ( t + Δ t ) - {F}_{m} ( t ) \frac{{Q}_{o} ( t + Δ t )}{{Q}_{o} ( t )} \cdot \frac{{F}_{o} ( t + Δ t ) - {F}_{m} ( t + Δ t )}{{F}_{o} ( t ) - {F}_{m} ( t )}}{1 - \frac{{Q}_{o} ( t + Δ t )}{{Q}_{o} ( t )} \cdot \frac{{F}_{o} ( t + Δ t ) - {F}_{m} ( t + Δ t )}{{F}_{o} ( t ) - {F}_{m} ( t )}}$$

$${F}_{E} ( t ) \approx \frac{{Q}_{o} ( t ) {F}_{m} ( t + Δ t ) ( {F}_{o} ( t ) - {F}_{m} ( t ) ) - {Q}_{o} ( t + Δ t ) {F}_{m} ( t ) ( {F}_{o} ( t + Δ t ) - {F}_{m} ( t + Δ t ) )}{{Q}_{o} ( t ) ( {F}_{o} ( t ) - {F}_{m} ( t ) ) - {Q}_{o} ( t + Δ t ) ( ( {F}_{o} ( t + Δ t ) - {F}_{m} ( t + Δ t ) )}$$

This expression may be used to determine an exhaled gas flow 13′ parameter such as the expired fraction of oxygen, carbon dioxide, nitrogen, helium and/or an anaesthetic agent such as sevoflurane.

In some configurations, a correction or compensation may be applied to equation (4) to obtain a better estimate of the parameter (F_{E}) of the gas flow component in the exhaled gas flow 13′. For example a correction or compensation may be applied to equation (4) to account for the assumption that the composite gas outflow and the proportion of the apparatus gas flow exiting the mouth to be approximately constant being not true. Such a correction or compensation may for example take into account a ratio of the mouth flow to patient interface flow as a function of patient interface flow rate, as described in the applicant's publication WO2017187391 or US2019/0150831, which are incorporated herein by reference in their entirety.

For measurements of CO₂, Fₒ(t)=Fₒ(t+Δt)˜0 and so equation (4) reduces to:

$${F}_{E} ( t ) ∼ \frac{{Q}_{o} ( t + Δ t ) {F}_{m} ( t ) {F}_{m} ( t + Δ t ) ) - {Q}_{o} ( t ) {F}_{m} ( t + Δ t ) {F}_{m} ( t )}{{Q}_{o} ( t + Δ t ) {F}_{m} ( t + Δ t ) - {Q}_{o} ( t ) {F}_{m} ( t )} \qquad ( 5 )$$

$${F}_{E} ( t ) ∼ {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{o} ( t ) {F}_{m} ( t )}$$

Above are expressions for oxygen and carbon dioxide in terms of known/measured quantities, Fₘ(t), Qₒ(t), Fₘ(t+Δt), Qₒ(t+Δt). The found F_{E}CO₂ can recover the carbon dioxide waveform seen in FIG. 2.

In summary, but without limitation, the apparatus is operated to oscillate the flow being administered through a patient interface, preferably a non-sealing nasal interface (cannula) (preferably within each breath). Doing this changes the gas fraction and allows the effect of the change in gas fraction to be removed from the underlying expired gas signal. Interpolation can be used to recover the waveform and value of the desired gas component in the patient exhaled gas flow.

The frequency at which flow is oscillated is configured to achieve the outcome of this invention. Oscillations must be sufficiently rapid such that the assumptions about Q_{E}, k and F_{E} hold and rapid enough that an interpolation can be made between the samples to sufficiently recover the waveform. The actual frequency required will depend on the patient's breathing frequency but will typically be in the range between 1-100 Hz. The oscillating frequency is preferably higher than the patient's breathing frequency or an average patient's breathing frequency (dependent on whether the patient is an adult or an infant). In an embodiment, the frequency may be approximately 5 Hz. In one possible embodiment, the controller could receive input (e.g. via a sensor—either directly, or indirectly via a person who e.g. reads a sensor) on the patient's breathing frequency. The controller can determine a suitable oscillating frequency from that input. In another possible embodiment, the controller could receive input from a user about the oscillating frequency and/or patient breathing frequency based on a patient's known breathing frequency, and the controller can determine a suitable oscillating frequency from that input.

With reference to FIG. 3A, if a continuous sinusoidal flow waveform was used in the apparatus (instead of stepping between flows) then this may allow for a lower frequency of oscillation. In the embodiment described above, each time step in flow enables collection of a data point. In the sinusoidal flow embodiment, each time the sine wave causes a measurable difference in CO₂ output, another data point can be gathered by the apparatus. This oscillating, sinusoidal embodiment also has a good signal to noise ratio.

An example scenario utilising this method relates to procedural sedation where a patient can be shallowly breathing. In this scenario, flow may be oscillating or otherwise varying (e.g. continuously or repetitively stepped)—between flow rates of e.g. between about 70 to 40 LPM (this may look like flows in FIG. 3A), over a period of time. When repetitively stepped, the flow rate in each step might be a single step change in flow rate, or a continuous/discrete change of various flow rates from one flow rate to the other. The expired fraction of carbon dioxide (or oxygen) can then be measured at the patient's mouth during high flow therapy at about 70 LPM and then again at about 40 LPM—this measurement is repeated as the flows are continually stepped up and down. This is a diluted measurement in the context of determining fraction of CO₂. The two flow rates (about 70 and about 40 LPM) and the expired fraction of CO₂ at these flow rates can be used in equation 5 to calculate an undiluted expired fraction of CO_{2.} This process is repeated over the period of time and the undiluted CO₂ measurements can be interpolated and presented to show the patient's expired CO₂ trace (this may look like the true waveform in FIG. 2). This expired CO₂ trace, and the end tidal CO₂ value that can be inferred or determined from it, is useful during procedural sedation as it can provide an indication of the levels of CO₂ in a patient that may have shallow breathing and thus, higher than expected CO₂. In this scenario, the diluted waveform which provides only an indication of gas exchange does not accurately provide this indication.

### 5. General Embodiment—Varying Apparatus Gas-Flow Gas Fraction

An alternative embodiment using a time-varying gas fraction in the apparatus gas flow is now described, referring to the apparatus described above. This is one example of time-varying gas proportion. Any control method can be implemented in the controller or any other suitable apparatus.

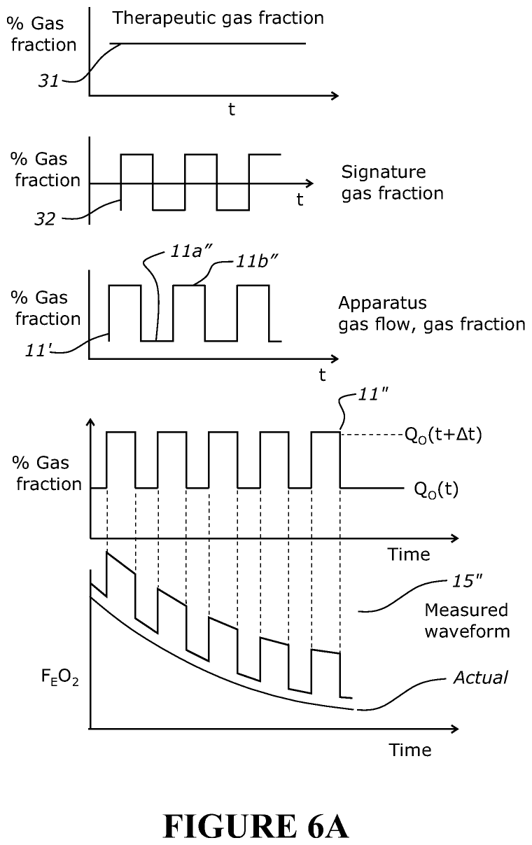

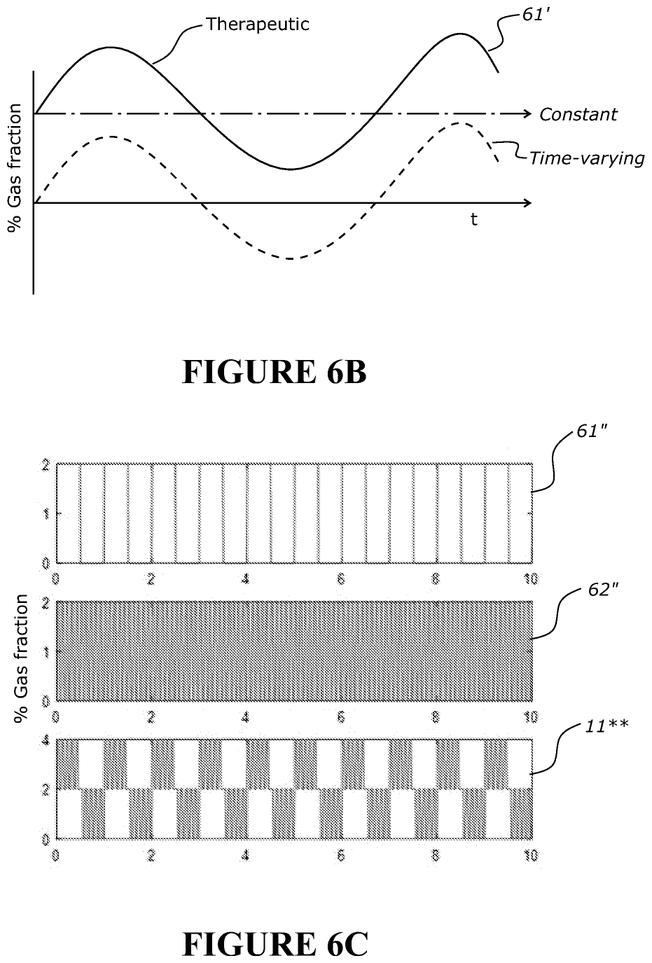

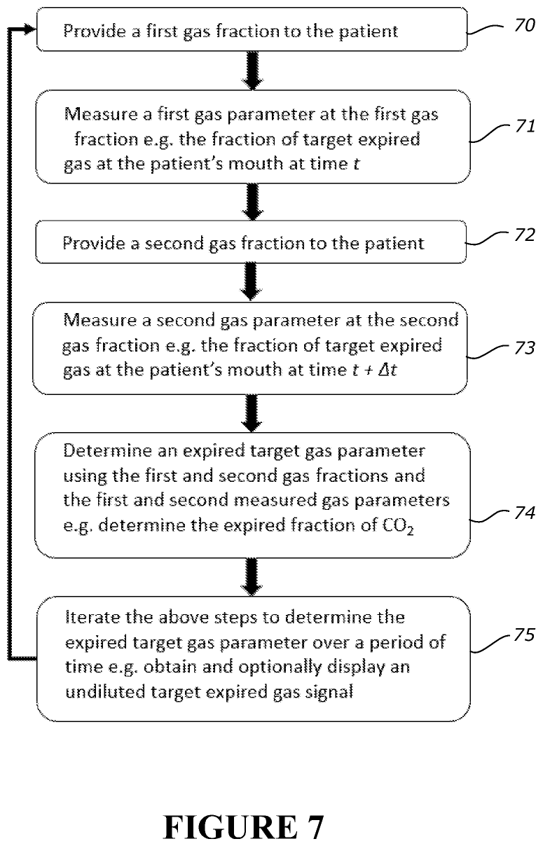

An embodiment is described, referring to the diagram and graphs of FIG. 6A and the flow diagram in FIG. 7. In general terms, determining a parameter is achieved by varying the gas fraction (over time) of the apparatus gas flow 11″ in a known manner and using knowledge of that time-varying gas fraction and information obtained from the composite gas outflow 15″ to obtain determine the parameter of the desired gas component in the actual exhaled gas flow 13″. The reference numeral 11″ is used for a varying apparatus gas flow with varying gas fraction, to distinguish from previously used reference numeral 11 that was used for an apparatus gas flow that does not vary, and reference numeral 11′ used for a varying flow rate gas flow. Likewise leak gas flow 12″, exhaled gas flow 13″, and composite gas outflow 15″ reference numerals are used where the apparatus gas flow is varying, instead of 11, 12, 13, 15 as used for the same parameters but where the apparatus gas flow does not vary.

In this embodiment, the gas fraction to determine is CO₂ fraction. But this is used by way of example only and should not be limiting. O₂ could be the gas fraction determined instead, and the same approach can be taken. However, with this embodiment, where the gas fraction in the apparatus gas flow 11″ is time-varied, it is the same type of gas that must be measured in the composite gas outflow 15″. So because O₂ (and not CO₂) is provided by the apparatus gas flow 11″, it is O₂ fraction that is time-varied in the apparatus gas flow 11″, and therefore it is O₂ fraction that is measured in the composite gas outflow 15″. But because CO₂ fraction is the desired gas parameter of interest, this is derived from the O₂ fraction that is measured, as will be described later. The modified time-varying gas flow could be provided at all times, or optionally just during expiration of the patient, so as to reduce any influence on respiratory support the signature might have.

As shown in FIG. 6A, apparatus gas flow 11″ with time-varying gas fraction (preferably O₂ fraction, but could work with other gases being provided) is provided to the patient by the respiratory apparatus 10. This apparatus gas flow with a time-varying gas fraction now comprises at least two gas fraction components. First is a therapeutic gas fraction component 61 according to what is required by therapy. Second, is a signature (time-varying) gas fraction 62 component that varies over time and modifies/modulates the therapeutic gas fraction, over and above what is required by therapy (including any time-varying of the gas fraction that might be required for the therapy) but in a manner that may not impact on the efficacy of the therapy provided by the apparatus gas flow. The two components add together to provide the overall time-varying apparatus gas flow gas fraction 11″. This is an explanation of the components of the time-varying gas fraction, but not necessarily an explanation of the how the time-varying gas fraction is achieved. Any suitable apparatus for varying a gas source to obtain a time varying gas fraction with the characteristics above could be implemented.

In one embodiment, there are two proportional valves that operate 180 degrees out of phase. As one opens, the other closes. One controls O₂ fraction in the apparatus gas flow, and the other controls Air fraction in the gas flow, but together keeping the total gas flow rate constant. In another alternative, a single proportional valve is used with an impeller where the proportional valve controls an O₂ fraction and the impeller controls the flow rate.

In some embodiments, the single proportional valve may be used before or after the impeller. Where the single proportion valve is used before the impeller, the proportional valve controls the O₂ fraction into the inlet of the impeller along with the ambient air. In some embodiments, more than one proportional valve may be used with an impeller and may be positioned anywhere in the system with respect to the impeller. The controller 19 can control the proportional valve(s) to operate as required to achieve the time-varying gas proportion as described herein.

As described above, the therapeutic gas fraction might be a constant gas fraction, but could also have time-varying gas fraction component itself (that is, a varying gas flow fraction with various time-varying fraction components). For example, as shown in FIG. 6B, the therapeutic gas fraction itself comprises multiple components 31″, including a constant (e.g. bias) component, and a time-varying component, which together add up to a time-varying component. (herein after, any reference to varying gas fraction means time-varying, even if not stated, as context allows). This can then be added to the signature gas fraction 62 (that is, the time-varying therapeutic fraction 61′ is modified/modulated by the signature gas fraction 62) to create an apparatus gas flow 11″. The apparatus gas flow with a time-varying gas fraction component 11″ preferably may have a non-varying flow rate.

In FIG. 6C is yet another example of a therapeutic gas flow 60″ with a time-varying gas fraction component—this time a square wave. (the therapeutic gas flow could also have a time-varying fraction as shown in FIG. 3B and described previously.) Also shown is a square wave signature gas fraction 62″ leading to the time-varying apparatus gas flow 11**.

The signature gas fraction might, for example, simply have a gas fraction that varies from a first gas fraction to a second gas fraction over time, but alternatively it could have any sort of varying gas fraction over time, such as an oscillating gas fraction or any other time varying gas fraction, be it periodic (regular or irregular), non-periodic, random, non-repeating or the like. For example, the signature gas fraction may be in the form of a square wave as shown in FIG. 6A. The signature gas fraction could also be a step function, sawtooth, sine wave, or a more complex random, repeating or non repeating function, or any other option where it varies over time between at least two different gas fractions. The signature gas fraction component adds to (modifies/modulates) the therapeutic gas fraction component to provide the varying apparatus gas fraction. Or it could be a combination of one or more waves, such as sine waves with various magnitudes and frequencies. Therefore, varying gas fraction means any gas fraction that changes at least once over time. The gas fraction of the apparatus gas flow will vary and comprise a therapeutic gas fraction (which may be constant, or itself might be varying, and therefore comprising various gas fraction components itself) and a signature gas fraction providing an additional component to vary the therapeutic gas fraction of the therapeutic gas flow. Preferably, the frequency of the signature gas fraction (where it is repeating) or the time period over which it changes (if it is not repeating) is higher than the frequency of the breath of the patient (“breathing frequency”) and/or is higher than the frequency of any variation of the therapeutic gas fraction component. Although, this is not essential. Again, this is an explanation of the components of the time-varying signature gas fraction component, but not necessarily an explanation of how the time-varying gas fraction component is achieved. It could be achieved in numerous ways, such as described earlier for varying flow rate.

As an example, FIG. 6A shows a varying gas fraction of an apparatus gas flow which comprises the gas fraction generated for therapeutic purposes 61, and the time-varying (signature) component 62. The time-varying signature component is a square wave function, which provides a regular repeating periodic gas fraction. When the leak gas flow 12″ adds to the exhaled gas flow 13″ of the patient, this creates a composite gas outflow 15″ that has the signature gas component 62 as a component see e.g. FIG. 3A. When measuring the gas parameter in the composite gas outflow (see bottom of FIG. 6A), the time-varying gas fraction of the apparatus gas flow 11″ affects the gas parameter in the composite gas outflow 15″, and becomes apparent in the measurement of the gas parameter. In this case the gas fraction being measured in the composite gas outflow 15″ is O₂, so the bottom graph of FIG. 6A shows O₂ fraction versus time.

The time-varying component 62 in the gas fraction of the apparatus gas flow 11″ in combination with measurement of the parameter of gas component of the patient composite gas outflow 15″ over time of the composite gas outflow parameter can be utilised to:
a) determine the effect the apparatus gas flow has on the parameter of the exhaled gas flow 13″, and/or
b) determine the gas parameter of the actual exhaled gas flow 13″. In this case, estimating O₂ proportion (in this case O₂ fraction) in the exhaled gas flow 13″ and then from that finding CO₂ proportion (in this case CO₂ gas fraction) in the exhaled gas flow 13″.

This determining of a) and/or b) is achieved by any suitable means, such as filtering, interpolating or extrapolating the composite gas outflow 15″ to obtain the gas flow parameter of the exhaled flow (that is, O₂ fraction in this example), modelling the gas flow parameter from the patient gas outflow 15″, calculating or otherwise determining the gas flow parameter from the composite gas outflow. Providing a signature gas fraction 62 to vary the apparatus gas-flow gas fraction 11″ changes the gas fraction (or other parameter being measured) in the composite gas outflow 15″ and allows the effect of the change in gas fraction to be removed from the underlying expired gas signal (exhaled gas flow 13″), in a suitable manner, either directly or indirectly. Interpolation can be used to recover the waveform and value of the expired gas, as one example. As another example, a measure of the apparatus gas flow gas fraction 11″ at two time points and the parameter of the gas fraction component in the composite gas outflow 15″ at the same two time points can be used to determine the parameter of the gas component in the exhaled flow. As an example, the proportion of O₂ in the exhaled gas flow 13″ can be determined by measuring/knowing the apparatus gas flow O₂ fraction at two times, and the O₂ fraction in the composite gas outflow at the same times. This can be repeatedly carried out at other times as the apparatus gas flow O₂ fraction varies over time. Other examples are possible too. As another example, in FIG. 6A, the actual gas flow parameter (O₂ fraction) can be extrapolated from the measurement, as shown. The CO₂ gas fraction in the exhaled gas flow 13″ can be determined from the O₂ fraction in the exhaled gas flow 13″, in a manner described later.

An apparatus and method for achieving the determination as described above will be described with reference to FIGS. 4 and 7. The apparatus has been described previously in relation to FIG. 4, and need not be described again. However, for clarity, the apparatus controller can operate the apparatus to control the oxygen fraction of the apparatus gas flow 13″. The controller can do this in any suitable way by operating the flow controller, the O₂ source, the air source, and any valves, motor, modulators or other apparatus that can be used to control oxygen fraction.

FIG. 7, which shows a method of operating a respiratory apparatus 10 with a sensor(s) 14 and controller 19 as per described above. The following steps can be undertaken. A time varying apparatus gas flow 11″ comprising a time-varying gas fraction (in this case O₂ fraction) with a signature is provided to the patient. In one variation, the time-varying fraction comprises at least a therapeutic gas fraction (O₂ ) component 61. The therapeutic gas fraction is modified/modulated by (e.g. in this case by adding) a signature gas fraction component 62 that varies over time to create an apparatus gas flow 11″ with a time-varying gas fraction with at least a second gas fraction (at a later time e.g. 11b″) that is different to the first gas fraction (at the earlier time e.g. 11a″). This could be a simple step change as in FIG. 6A, or as noted above a more complex time-varying gas fraction waveform, such as a square wave, sinusoidal wave or any other wave described herein or envisaged. The “modified” apparatus gas flow 11″ is provided to the patient. The first gas fraction 11a″ of the apparatus gas flow 11″ is provided to the patient, step 70, and the second gas fraction 11b″ of the apparatus gas flow 11″ is provided to the patient sometime later, step 72. The gas fractions are preferably known, or alternatively measured. At the same time, the respiratory apparatus 10 is monitoring the composite gas outflow 15″ over time, which is a combination of the leak gas flow 12″ (with a time-varying gas fraction) and the exhaled gas flow 13″. This comprises at least measuring a first parameter (in this case O₂ fraction) at the time of the first gas fraction 11a″ of the composite gas outflow and sometime later correlating to the first gas fraction 11a″ being provided, step 71, and the same gas parameter (O₂ fraction) of the composite gas outflow 15″ at the second gas fraction 11b″ at a later point in time, step 73.

The apparatus 10 could compensate for a delay in providing the first gas fraction 11a″ and the measurement of the first parameter in the composite gas outflow 15″ affected by the first gas fraction, for example in the case of side stream sampling, a correction of “X” seconds could be applied.

The parameter (O₂ gas fraction) of the composite gas outflow 15″ is measured at or near the mouth and/or nose (“proximate”) of the patient using the sensor 14. Only one gas parameter (the target gas parameter of interest) need be measured. The gas parameter (O₂ fraction) in the exhaled gas flow 13″ is then determined using (preferably known, but optionally measured) the first and second gas fractions (e.g. O₂ fractions) of the apparatus gas flow 11″ and the first and second measured gas parameters (e.g. O₂ fraction) of the composite gas outflow 15″ at the first and second points in time, step 74. The CO₂ gas fraction in the exhaled gas flow 13″ can then be derived from the O₂ fraction of the exhaled gas flow 13″ as will be described below.

Steps 70 to 74 can be repeated continuously/periodically, step 75, to obtain a measure of the gas parameter (O₂ fraction) of the exhaled gas flow 13′ over time as the apparatus gas flow 11″ varies its gas fraction 11a″, 11b″ over time due to the varying signature gas fraction 62. The parameter of the gas component can be measured in the composite gas outflow 15″ during expiration phase of the patient breathing. While simply a first 11a″ and second gas 11b″ fraction in the apparatus gas flow 11″ could be used, in practice the signature gas fraction 62 is likely to be varying continuously, or at least periodically/discretely over time and the composite gas outflow 15″ can be measured continuously or periodically (e.g. at a sampling rate) to get continuous or periodic measures of the exhaled gas flow parameter, which can then be displayed via a graph or display to give a real-time measure of the exhaled gas flow parameter.

This embodiment may be particularly useful for the pre-oxygenation scenario described where the patient is likely to be spontaneously breathing. Varying oxygen fraction rather than flow rate may be more comfortable for the patient if the patient is conscious and/or awake.

The controller may also use a phase-locked loop which synchronises the phase of the varying gas flow and the measured gas parameters.

### 6. Exemplary Embodiment—Varying Apparatus Gas-Flow Gas Fraction

One exemplary embodiment, will now be described with reference to the Figures, using the apparatus, and control methods described above. Like for the varying apparatus gas-flow flow rate embodiment, in this embodiment, the medical professional desires to monitor exhaled CO₂, O₂, nitrogen, helium and/or an anaesthetic agent such as Sevoflurane. The description of the purpose and arrangement of apparatus from that embodiment (exemplary embodiment—varying apparatus gas-flow flow rate) applies here also. The embodiment utilises a flow rate without a time-varying signature. The flow rate may be a set flow rate which does not vary. Any control method can be implemented in the controller or any other suitable apparatus.

For illustrative purposes, the gas fraction to determine is O₂ and/or CO₂ fraction. But this is used by way of example only and should not be limiting. Other gases could be the target gas instead—such as Nitrogen, Helium and/or an anaesthetic agent such as Sevoflurane. In such cases another suitable sensor is used to sense the target gas in the composite gas outflow—that is a suitable sensor to sense Nitrogen, Helium and/or an anaesthetic agent such as Sevoflurane etc.

In a first step, O₂ gas fraction is determined in the exhaled gas flow. Then, in a second optional step, CO₂ gas fraction in the exhaled gas flow can then also be determined from the O₂ gas fraction in the exhaled gas flow. However, with this embodiment, where the gas fraction in the apparatus gas flow 11″ is time-varied, it is the same type of gas that is measured in the composite gas outflow 15″. So because O₂ (and not CO₂) is provided by the apparatus gas flow 11″, it is O₂ fraction that is time-varied in the apparatus gas flow 11″, and therefore it is O₂ fraction that is measured in the composite gas outflow 15″. CO₂ fraction may then be derived from the O₂ fraction that is measured, as will be described later.

The apparatus is operated to provide an apparatus gas flow 11″ with a time-varying gas fraction (O₂ gas fraction) as previously described with a therapeutic gas fraction component 61 and a signature (time-varying) gas fraction component 62. In this example, a constant therapeutic gas fraction 61 is provided and is added to a square wave signature gas fraction component 62 to produce the apparatus gas flow 11″ with time-varying gas fraction, as shown in FIG. 6A. In some embodiments, the signature gas fraction component 62 may be added to a constant therapeutic gas fraction component 61 to produce the apparatus gas flow 11″ with a time-varying gas fraction, as shown in FIG. 6A. The controller 19 or a user operates the flow source 10 to provide an apparatus gas flow 11″ with a gas fraction. The controller 19 may be programmed with the desired apparatus gas flow 11″ with a varying gas fraction and creates the varying gas fraction to provide the required variation in the gas fraction over time.

In use, the patient breathes in the apparatus gas flow 11″ and exhales a gas flow 13″. The exhaled gas flow 13″ combines with the leak gas flow 12″ to produce a composite gas outflow 15″ as shown in FIG. 6A. The last graph showing F_{E}O₂ is for the expiratory phase.

The sensor 14 measures the O₂ fraction in the composite gas outflow 15″ and passes this information to the controller 19. The output of the sensor 14 measuring the parameter of the composite gas outflow 15″ is represented as the waveform 15″ shown in FIG. 6A (which shows the gas fraction during expiration). The controller then needs to process that output to determine the actual O₂ fraction in the exhaled patient gas flow 13″. From that, it can then derive the actual CO₂ fraction in the exhaled gas flow 13″.

The fraction of exhaled gas component, e.g. O₂ as a percentage of the total gas exhaled per volume can be determined from the composite gas outflow 15″ using equations that utilise knowledge of the change in gas fraction of the time-varying apparatus gas flow. This determination can be obtained based on the premise that the fraction of exhaled gas (component) can be determined from the known/measured quantities:

Fₘ(t), volume fraction of the gas component measured in the patient composite gas outflow 15′ from the patient (this is the measured CO₂/O₂ fraction parameter of the composite gas outflow 15′, preferably measured by the sensor 14) at time t.

Fₘ(t) is preferably measured at the mouth of the patient when the patient's mouth is open and/or or the nose if the patient's mouth is closed.

Fₒ(t), gas fraction of the apparatus gas flow 11′ provided from the respiratory apparatus to the patient (apparatus gas flow rate) at time t.

Fₘ(t+Δt), volume fraction of the gas component measured in the patient composite gas outflow 15′ (this is the measured CO₂/O₂ fraction parameter of the composite gas outflow, preferably measured by the sensor 14) at time t+Δt. Fₘ(t+Δt) is preferably measured at the mouth of the patient when the patient's mouth is open or the nose if the patient's mouth is closed.

Fₒ(t+Δt), gas fraction of apparatus gas flow 11′ provided from the respiratory apparatus to the patient (apparatus gas flow rate), at time t+Δt.

For varying gas fraction, equation (6) can be used, which is derived from equation (4)

If the gas fraction is varied, but the flow rate Qₒ is constant (i.e. not varying), then

Qₒ(t+Δt)=Qₒ(t)

so the flow terms cancel out. This results in the simplified equation:

$${F}_{E} ( t ) = \frac{{F}_{m} ( t + Δ t ) ( {F}_{o} ( t ) - {F}_{m} ( t ) ) - {F}_{m} ( t ) {F}_{o} ( t + Δ t ) - {F}_{m} ( t + Δ t ) )}{( {F}_{o} ( t ) - {F}_{m} ( t ) ) - ( ( {F}_{o} ( t + Δ t ) - {F}_{m} ( t + Δ t ) )} \qquad ( 6 )$$

Equation (6) provides F_{E} as a function of:

Fₒ(t),Fₒ(t+Δt),Fₘ(t+Δt),Fₘ(t)

This equation allows the fraction of the expired gas in the exhaled gas flow 13 (in this case O₂) to be recovered.

Note that:
1. the assumption that:

$$\frac{{Q}_{E} ( t + Δ t )}{k ( t + Δ t )} ∼ \frac{{Q}_{E} ( t )}{k ( t )}$$

used in deriving (4) may become more accurate when only the fraction is being varied (i.e. varying the flow might change this ratio but it is unlikely that varying the gas fraction in the apparatus flow will)
2. The apparatus can vary O₂ fraction just during expiration to avoid diluting or otherwise interfering with oxygen delivered to the patient.

In addition, it is possible to go further and determine the CO₂ gas fraction in the exhaled gas from the O₂ gas fraction in the exhaled gas flow.

Knowledge of F_{E}(t) for oxygen allows determination of F_{E}(t) for carbon dioxide as follows:

Assuming that Fₒ=0 for carbon dioxide (i.e. that the fraction of carbon dioxide in the gas from the respiratory apparatus is negligible) then equation (3) can be rewritten as:

$$\frac{{Q}_{E} ( t )}{k ( t )} = {Q}_{o} ( t ) \frac{- {F}_{m} ( t )}{{F}_{m} ( t ) - {F}_{E} ( t )} \qquad ( 7 )$$

Rearranging for F_{E}(t) yields:

$${F}_{ECO 2} = {F}_{mCO 2} ( 1 + \frac{k {Q}_{o}}{{Q}_{E}} ) \qquad ( 8 )$$

Where FmCO2 should be the volume fraction of CO2 in the patient composite gas outflow 15′ from the patient

(See heading below for a derivation of equation (8))

So FeCO2 is now expressed in terms of known quantities, in that kQₒ/Q_{E} is now known from equation (3) since Fₑ(t) for oxygen is known. So CO₂ fraction in the exhaled gas flow 13″ can be determined from O₂ fraction in the exhaled gas flow 13″.

The O₂ fraction in the composite gas outflow 15″ is measured by the sensor 14, and the gas fraction of the apparatus gas flow 11″ is known (or can be measured). So, by measuring the O₂ fraction in the composite gas outflow 15′ using sensor 14 at two (or more) different times, and knowing/measuring the gas fraction of the apparatus gas flow 13″ at the two (or more) different times, the O₂ fraction in the exhaled gas flow 13″ and then the CO₂ fraction in the exhaled gas flow 13″ can be determined using equation (6) and (8).

In practice, the O₂ fraction will be measured continuously or periodically/discretely to go with a real-time output, which can then be used in combination with knowledge/measurement of the apparatus gas flow 11″ that is also sampled continuously or periodically to determine a real-time CO₂ (from O₂ ) fraction of the exhaled gas flow 13″. This can be output as a graph, digital readout or the like on the display. In practice, the gas fraction will vary continuously, or discretely but periodically or regularly over a time period to provide a plurality of data points. Therefore, by varying the gas fraction at least once, the difference between a first 11a″ and second gas 11b″ fraction can be used. The controller will therefore measure the gas parameter at suitable time points and then for each measurement calculate F_{E}.

Some general comments on this embodiment follow:
The gas measured in the composite gas outflow 15″ (i.e. the gas of interest) is O₂ but this is used to ultimately obtain an determination of CO₂ fraction in the exhaled gas flow 13″.
The time-varying parameter comprises a gas fraction, preferably O₂
The time-varying gas fraction comprises a therapeutic gas fraction component and a time-varying gas fraction component
The time-varying gas fraction varies between 21% to 100%
Preferably, the method is applied to a spontaneously breathing patient
The time-varying gas fraction is optionally applied throughout the respiratory cycle of the patient. Alternatively, the time-varying gas fraction is applied during the expiratory phase of the patient
In one variation, the method comprises determining if the O₂ fraction has reached a pre-determined threshold (useful for indicating the end of the pre-oxygenation phase)
Once O₂ fraction is determined for the exhaled gas flow 13″, as an optional addition, a CO₂ fraction in the exhaled gas flow 13″ may be derived.

As demonstrated above, once O₂ fraction is known in the exhaled gas flow (as determined by measuring the O₂ in the composite gas out flow and using e.g. equation (6)), the CO₂ gas fraction can be determined. It is not essential to determine the O₂ gas fraction in this manner using equation (6). Rather, it could be determined in another manner—such as through a sensor or some other means for determining O₂ gas fraction in the exhaled gas flow. Once that is done, CO₂ gas fraction in the exhaled gas flow can be determined by equation (8).

### 6.1 Derivation of Equation (8)

We use equation (3) to derive F_{ECO2} from the F_{EO2}:

$$\frac{{Q}_{E} ( t )}{k ( t )} = {Q}_{o} ( t ) \frac{{F}_{o} ( t ) - {F}_{m} ( t )}{{F}_{m} ( t ) - {F}_{E} ( t )} \qquad ( 3 / 7.1 )$$

The O₂ version of this equation is:

$$\frac{{Q}_{E} ( t )}{k ( t )} = {Q}_{o} ( t ) \frac{{F}_{oO 2} ( t ) - {F}_{mO 2} ( t )}{{F}_{mO 2} ( t ) - {F}_{EO 2} ( t )} \qquad ( 7.2 )$$

And the CO₂ version is:

$$\frac{{Q}_{E} ( t )}{k ( t )} = {Q}_{o} ( t ) \frac{{F}_{oCO 2} ( t ) - {F}_{mCO 2} ( t )}{{F}_{mCO 2} ( t ) - {F}_{ECO 2} ( t )} \qquad ( 7.3 )$$

But F_{oCO2}(t)˜0 for all t (i.e. the amount of CO₂ being delivered by the high flow system is negligible) so equation (7.3) reduces to:

$$\frac{{Q}_{E} ( t )}{k ( t )} = - {Q}_{o} ( t ) \frac{{F}_{mCO 2} ( t )}{{F}_{mCO 2} ( t ) - {F}_{ECO 2} ( t )} \qquad ( 7.4 )$$

Setting equation (7.4) equal to equation (7.2) yields:

$${Q}_{o} ( t ) \frac{{F}_{oO 2} ( t ) - {F}_{mO 2} ( t )}{{F}_{mO 2} ( t ) - {F}_{EO 2} ( t )} = - {Q}_{o} ( t ) \frac{{F}_{mCO 2} ( t )}{{F}_{mCO 2} ( t ) - {F}_{ECO 2} ( t )} \qquad ( 7.5 )$$

Qₒ (t) cancels so equation (7.5) can be reduced to:

$$\frac{{F}_{oO 2} ( t ) - {F}_{mO 2} ( t )}{{F}_{mO 2} ( t ) - {F}_{EO 2} ( t )} = - \frac{{F}_{mCO 2} ( t )}{{F}_{mCO 2} ( t ) - {F}_{ECO 2} ( t )} \qquad ( 7.6 )$$

F_{EO2} can be found as described above using equation (6) so we can rearrange equation (7.6) to find Fe_{CO2} in terms of known quantities:

$$\frac{{F}_{ECO 2} ( t ) - {F}_{mCO 2} ( t )}{{F}_{mCO 2} ( t )} = \frac{{F}_{mO 2} ( t ) - {F}_{EO 2} ( t )}{{F}_{oO 2} ( t ) - {F}_{mO 2} ( t )} \qquad ( 7.7 )$$

$${F}_{ECO 2} ( t ) = {F}_{mCO 2} ( t ) \frac{{F}_{mO 2} ( t ) - {F}_{EO 2} ( t )}{{F}_{oO 2} ( t ) - {F}_{mO 2} ( t )} + {F}_{mCO 2} ( t ) \qquad ( 7.8 )$$

$${F}_{ECO 2} ( t ) = {F}_{mCO 2} ( t ) [ 1 + \frac{{F}_{mO 2} ( t ) - {F}_{EO 2} ( t )}{{F}_{oO 2} ( t ) - {F}_{mO 2} ( t )} ] \qquad ( 7.9 )$$

So varying the oxygen concentration in the high flow allows to measure both FeO2 via equation (6) and FeCO2 (via equation (7.9), (8)).

### 7. Other Variations

The following variations/additions are possible:
The apparatus may be used with any gas sampling device—side stream or mainstream sampling.
The controller can control flow rates, fractions, partial pressures or the like based on received information. Any of the information could be received from a person via user input via the user interface rather than from a sensor. Also, any changes the controller make could be determined by the controller, or based on input received from a user via user input via the user interface.
The F_{E}O₂ may also be measured in the same way as F_{E}CO₂ has been described to be measured but by measuring patient O₂ fraction.
The embodiments have been described for finding the exhaled gas flow gas parameter for one gas, e.g. CO₂ or O₂. But, in the case where a clinician might be monitoring more than one gas parameter (e.g. monitoring O₂ fraction to assess pre-oxygenation and also monitoring CO₂ to assess breathing) the embodiments described above can be applied to finding the correct exhaled gas flow parameter for all gas parameters of interest.
The time-varying gas fraction embodiment could be implemented as a time-varying gas partial pressure embodiment by use of different sensors and processing. Those skilled in the art will understand the relationship between gas fraction and gas partial pressure, and could adapt accordingly.

The embodiments above are just some examples and should not be considered limiting. In more general terms, any method and/or apparatus that can time vary a parameter in the apparatus gas flow 11 can be used to determine a gas fraction in the exhaled gas flow 13 based on the concepts embodied in the model above. As an example, by finding:

(using time-varying flow rate) F_{E} of the gas as a function of:

Qₒ,Qₒ(t+Δt),Fₘ(t+Δt),Fₘ(t)

or
(using time-varying gas fraction) F_{E} of the gas as a function of:

Fₒ(t),Fₒ(t+Δt),Fₘ(t+Δt),Fₘ(t)
Embodiments have referred to O₂ and CO₂, but other gas parameters can also be determined using similar methods and apparatus, where for example, the sensor for the composite gas outflow is a sensor to detect the target gas parameter of interest in the exhaled gas flow.

1. A method of determining a parameter of a gas present in an exhaled gas flow comprising:
providing an apparatus gas flow with a time-varying parameter to a patient,
measuring a parameter of the gas present in a composite gas outflow from the patient,
and
determining the parameter of the gas present in the exhaled gas flow using the measured parameter of the gas present in the composite gas outflow and the time-varying parameter.

2. An apparatus for providing an apparatus gas flow and determining a parameter of an exhaled patient gas flow, comprising:
a flow source,
a sensor for sensing a composite gas outflow, and
a controller,
wherein the apparatus is configured to:
provide an apparatus gas flow with a time-varying parameter,
determine a parameter of a gas present in a composite gas outflow from the patient, the composite gas outflow comprising:
leak gas flow from apparatus gas flow, and
exhaled gas flow from a patient with the gas,
and
determine the parameter of the gas present in the exhaled gas flow using the determined gas parameter and the time-varying parameter.

3. A method or apparatus according to claim 1 or 2 wherein the time varying parameter is one or more of:
flow rate of the apparatus gas flow, or
gas proportion, where optionally the gas proportion is:
a fraction of a gas present in the apparatus gas flow,
a partial pressure of a gas present in the apparatus gas flow.

4. A method or apparatus according to claim 1, 2 or 3 wherein the gas proportion is:
gas fraction, preferably O₂ fraction; or
gas partial pressure, preferably O₂ partial pressure.

5. A method or apparatus according to any preceding claim comprising providing the apparatus gas flow during an anaesthetic procedure.

6. A method or apparatus according to any preceding claim wherein the apparatus gas flow is provided via a non-sealing patient interface, preferably a non-sealing cannula.

7. A method or apparatus according to any preceding claims where the apparatus gas flow is a high flow gas flow.

8. A method or apparatus according to any preceding claim further comprising humidifying the apparatus gas flow.

9. A method or apparatus according to any preceding claim wherein determining the parameter of the gas present in the exhaled gas flow using the measured parameter of the gas present in the composite gas outflow comprises measuring just one gas and just the time varying parameter, the time varying parameter being flow rate.

10. A method of determining a parameter of a gas present in an exhaled gas flow comprising:
providing an apparatus gas flow with a time-varying flow rate to a patient,
determining a parameter of the gas present in a composite gas outflow from
the patient, the patient composite gas outflow comprising:
leak gas flow from apparatus gas flow, and
exhaled gas flow from a patient with the gas,
and
determining the parameter of the gas present in the exhaled gas flow using the determined parameter of the gas present in the composite gas outflow and the time-varying flow rate.

11. A method according to claim 10 wherein the apparatus gas flow with the time-varying flow rate comprises at least a first flow rate at a first time and a second flow rate at a second time, and
determining the parameter of the gas present in the exhaled gas flow using the determined parameter of the gas present in the composite gas outflow and the time-varying flow rate comprises:
using the determined parameter of the gas present in the composite outflow determined at the first flow rate and determined at the second flow rate.

12. A method according to claim 10 wherein the parameter comprises a fraction of the gas component in the exhaled gas flow.

13. A method or apparatus according to preceding claim wherein the gas is:
CO₂,
O₂,
Nitrogen,
Helium, and/or
Anaesthetic agent such as sevoflurane.
and/or
the sensor is configured to sense one or more of the following in the composite gas outflow:
CO₂,
O₂,
Nitrogen,
Helium, and/or
anaesthetic agent such as sevoflurane.

14. A method according to any one of claims 10 to 13 wherein the parameter of the gas present in the composite gas outflow is determined during an inspiration and/or expiration phase of the patient breathing.

15. A method according to any one of claims 10 to 14 wherein the parameter of gas present in an exhaled gas flow is gas fraction and determining the fraction of the gas (F_{E}) present in an exhaled gas flow using the determined parameter of gas present in the composite gas outflow and the time-varying flow rate comprises using:
$${F}_{E} ( t ) = \frac{\begin{matrix}{Q}_{o} ( t ) {F}_{m} ( t + Δ t ) ( {F}_{o} ( t ) - {F}_{m} ( t ) ) - \\ {Q}_{o} ( t + Δ t ) {F}_{m} ( t ) ( {F}_{o} ( t + Δ t ) - {F}_{m} ( t + Δ t ) )\end{matrix}}{\begin{matrix}{Q}_{o} ( t ) ( {F}_{o} ( t ) - {F}_{m} ( t ) ) - \\ {Q}_{o} ( t + Δ t ) ( ( {F}_{o} ( t + Δ t ) - {F}_{m} ( t + Δ t ) )\end{matrix}} \qquad ( 4 )$$
Where
F_{E}(t) is the CO₂ or O₂ or other gas fraction in the exhaled patient gas flow (volume fraction of expired gas)
Fₘ(t), fraction of the gas component measured in the patient composite gas outflow at time t
Qₒ(t), flow rate of the apparatus gas flow provided to the patient (apparatus gas flow rate), at time t
Fₘ(t+Δt), fraction of the gas component measured in the patient composite gas outflow (e.g. this is the measured CO₂, O₂, Nitrogen, Helium and/or Anaesthetic agent such as sevoflurane fraction parameter of the composite gas outflow) at time t+Δt
Qₒ(t+Δt), flow rate of the apparatus gas flow provided to the patient (apparatus gas flow rate) at time t+Δt
Fₒ(t), volume fraction of the gas component in the apparatus gas flow 11′ coming from the respiratory apparatus, at time t and t+Δt.
Fₒ(t+Δt), volume fraction of the gas component in the apparatus gas flow 11′ coming from the respiratory apparatus, at time t+Δt.

16. A method according to any one of claims 10 to 15 wherein the parameter of gas present in an exhaled gas flow is gas fraction and determining the fraction of the gas (F_{E}) present in an exhaled gas flow using the determined parameter of gas present in the composite gas outflow and the time-varying flow rate comprises determining the gas fraction F_{E}(t) as a function of:
Qₒ(t),Qₒ(t+Δt),Fₘ(t+Δt),Fₘ(t)
where
Fₘ(t), volume fraction of the gas component measured in the patient composite gas outflow 15′ from the patient (this is the measured CO₂/O₂ fraction parameter of the composite gas outflow 15′, preferably measured by the sensor 14) at time t.
Fₘ(t) is preferably measured at the mouth of the patient when the patient's mouth is open and/or or the nose if the patient's mouth is closed.
Qₒ(t), flow rate of the apparatus gas flow 11′ provided from the respiratory apparatus to the patient (apparatus gas flow rate) at time t.
Fₘ(t+Δt), volume fraction of the gas component measured in the patient composite gas outflow 15′ (this is the measured CO₂/O₂ fraction parameter of the composite gas outflow, preferably measured by the sensor 14) at time t+Δt. Fₘ(t+Δt) is preferably measured at the mouth of the patient.
Qₒ(t+Δt), flow rate of apparatus gas flow 11′ provided from the respiratory apparatus to the patient (apparatus gas flow flow rate), at time t+Δt.

17. A method according to any one of claims 10 to 15 wherein the parameter of the gas present in the exhaled gas flow is gas fraction and the gas is preferably CO₂ and determining the gas fraction (F_{E}) in the exhaled gas flow using the determined parameter of gas present in the composite gas outflow and the time-varying flow rate comprises using:
$${F}_{E} ( t ) ∼ {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{o} ( t ) {F}_{m} ( t )} \qquad ( 5 )$$
Where
F_{E}(t) is the CO₂ and/or O₂ concentration in the exhaled patient gas flow (volume fraction of expired gas)
Fₘ(t), fraction of CO₂ measured in the composite gas outflow at time t
Qₒ(t), flow rate of the apparatus gas flow provided to the patient (apparatus gas flow flow rate) at time t
Fₘ(t+Δt), fraction of CO₂ measured in the composite gas outflow at time t+Δt
Qₒ(t+Δt), flow rate of the apparatus gas flow provided to the patient (apparatus gas flow flow rate) at time t+Δt

18. A method according to any one of claims 10 to 17 wherein the parameter of the gas present in the composite gas outflow from the patient is measured at or near the mouth and/or nose of the patient.

19. A method according to any one of claims 10 to 18 wherein the first and second flow rates are different flow rates.

20. A method according to any one of claims 10 to 19 wherein the first and second flow rates are high flow rates.

21. A method according to any one of claims 10 to 20 wherein the first and second flow rates are greater than or equal to about 0 L per minute, and preferably about or greater than about 20 L per minute, and more preferably between about 20 L per minute to about 90 L per minute.

22. A method according to any one of claims 10 to 21 wherein the time varying flow rate is an oscillation with a varying flow rate of greater than or equal to about 0 L per minute, and preferably about or greater than about 20 L per minute, and more preferably between about 20 L per minute to about 90 L per minute.

23. A method according to any one of claims 10 to 22 comprising providing the apparatus gas flow during an anaesthetic procedure.

24. A method according to any one of claims 10 to 23 wherein the apparatus gas flow is provided via a non-sealing patient interface, preferably a non-sealing cannula.

25. A method according to any one of claims 10 to 24 where the apparatus gas flow is a high flow gas flow.

26. A method according to any one of claims 10 to 25 further comprising humidifying the apparatus gas flow.

27. A method according to any one of claims 10 to 26 wherein determining the parameter of the gas present in the exhaled gas flow using the measured parameter of the gas present in the composite gas outflow comprising measuring just one gas and just the time varying parameter, the time varying parameter being flow rate.

28. A method of determining a parameter of a gas present in an exhaled gas flow comprising:
providing an apparatus gas flow with a time-varying gas proportion (for example gas fraction) to a patient,
determining a parameter of the gas present in a composite gas outflow from the patient, the patient composite gas outflow comprising:
leak gas flow from apparatus gas flow, and
exhaled gas flow from a patient with the gas,
and
determining the parameter of the gas present in the exhaled gas flow using the determined parameter of the gas present in the composite gas outflow and the time-varying gas proportion (for example gas fraction).

29. A method according to claim 28 wherein the apparatus gas flow with the time-varying gas fraction comprises at least a first gas fraction at a first time and a second gas fraction at a second time, and
determining the parameter of the gas present in the exhaled gas flow using the determined parameter of the gas present in the composite gas outflow and the time-varying gas fraction comprises:
using the determined parameter of the gas present in the composite outflow determined at the first gas fraction and determined at the second gas fraction.

30. A method according to claim 28 wherein the parameter comprises a fraction of the gas component in the exhaled gas flow.

31. A method according to any one of claims 28 to 30 wherein the gas is:
CO₂,
O₂,
Nitrogen,
Helium, and/or
Anaesthetic agent such as sevoflurane.

32. A method according to any one of claims 28 to 31 wherein the parameter of the gas present in the composite gas outflow is determined during an inspiration and/or expiration phase of the patient breathing.

33. A method according to any one of claims 28 to 32 wherein the parameter of gas present in an exhaled gas flow is gas fraction and determining the fraction of the gas (F_{E}) present in an exhaled gas flow using the determined parameter of gas present in the composite gas outflow and the time-varying gas fraction F_{E}(t) comprises determining the gas fraction as a function of:
Fₒ(t),Fₒ(t+Δt),Fₘ(t+Δt),Fₘ(t)
F_{E}(t) is the CO₂ and/or O₂ gas fraction in the exhaled patient gas flow (volume fraction of expired gas) at time t
Fₘ(t), fraction of CO₂ measured in the composite gas outflow at time t
Fₒ(t), gas fraction of the apparatus gas flow provided to the patient (apparatus gas flow gas fraction) at time t
Fₘ(t+Δt), fraction of CO₂ measured in the composite gas outflow at time t+Δt Fₒ(t+At), gas fraction of the apparatus gas flow provided to the patient (apparatus gas flow gas fraction) at time t+Δt

34. A method according to any one of claims 28 to 33 wherein the parameter of the gas present in the exhaled gas flow is gas fraction and the gas is preferably CO₂, O₂, Nitrogen, Helium and/or Anaesthetic agent such as sevoflurane and determining the gas fraction (F_{E}) in the exhaled gas flow using the determined parameter of gas present in the composite gas outflow and the time-varying gas fraction comprises using:
$${F}_{E} ( t ) = \frac{{F}_{m} ( t + Δ t ) ( {F}_{o} ( t ) - {F}_{m} ( t ) ) - {F}_{m} ( t ) ( {F}_{o} ( t + Δ t ) - {F}_{m} ( t + Δ t ) )}{( {F}_{o} ( t ) - {F}_{m} ( t ) ) - ( ( {F}_{o} ( t + Δ t ) - {F}_{m} ( t + Δ t ) )} \qquad ( 6 )$$
Where
F_{E}(t) is the CO₂ and/or O₂ concentration in the exhaled patient gas flow (volume fraction of expired gas)
Fm(t), fraction of CO₂ and/or O₂ or other gas measured in the composite gas outflow at time t
Fₒ(t), gas fraction of the apparatus gas flow provided to the patient (apparatus gas flow gas fraction) at time t
Fₘ(t+Δt), fraction of CO₂ and/or O₂ or other gas measured in the composite gas outflow at time t+Δt
Fₒ(t+Δt), gas fraction of the apparatus gas flow provided to the patient (apparatus gas flow gas fraction) at time t+Δt

35. A method according to any one of claims 28 to 34 wherein the parameter of the gas present in the composite gas outflow from the patient is measured at or near the mouth and/or nose of the patient.

36. A method according to any one of claims 28 to 35 wherein the first and second gas fractions are different gas fractions.

37. A method according to any one of claims 31 to 36 wherein
the gas is O₂, and
the method further comprises determining the CO₂ proportion present in the exhaled gas flow using the determined O₂ proportion and a function of:
F_{mCO2},k,Qₒ,Q_{E}
where
F_{mCO2} is the fraction of CO₂ measured in the patient composite gas outflow from the patient
k is the proportion of the apparatus gas flow that comes out through the patient's mouth (and (1−k) is the proportion that goes through the nose)
Qₒ is the flow rate of the apparatus gas flow.
Q_{E} is the flow rate of the patient exhaled gas flow.

38. A method according to any one of claims 31 to 36 wherein
the gas is O₂, and
the method further comprises determining the CO₂ proportion present in the exhaled gas flow using the determined O₂ proportion and
$${F}_{ECO 2} = {F}_{mCO 2} ( 1 + \frac{k {Q}_{o}}{{Q}_{E}} ) \qquad ( 8 )$$
where
F_{mCO2} is the fraction of CO₂ measured in the patient composite gas outflow from the patient k is the proportion of the apparatus gas flow that comes out through the patient's mouth (and (1−k) is the proportion that goes through the nose)
Qₒ is the flow rate of the apparatus gas flow.
Q_{E} is the flow rate of the patient exhaled gas flow.

39. An apparatus for providing an apparatus gas flow and determining a parameter of gas present in an exhaled patient gas flow, comprising:
a flow source,
a sensor for sensing a composite gas outflow, and
a controller,
wherein the apparatus is configured to:
provide an apparatus gas flow with a time-varying flow rate,
determine a parameter of a gas present in a composite gas outflow from the patient, the composite gas outflow comprising:
leak gas flow from apparatus gas flow, and
exhaled gas flow from a patient with the gas,
and
determine the parameter of the gas present in the exhaled gas flow using the determined parameter of gas present in the composite gas outflow and the time-varying flow rate.

40. An apparatus according to claim 39 further comprising a humidifier for humidifying the apparatus gas flow.

41. An apparatus according to claim 39 or 40 further comprising a non-sealing patient interface, and preferably a non-sealing nasal cannula to provide the apparatus gas flow to a patient.

42. An apparatus according to any one of claims 39 to 41 wherein the apparatus gas flow is a high flow gas flow.

43. An apparatus according to any one of claims 39 to 42 wherein the apparatus gas flow with the time-varying flow rate comprises at least a first flow rate at a first time and a second flow rate at a second time, and
determining the parameter of the gas present in the exhaled gas flow using the determined parameter of the gas present in the composite gas outflow and the time-varying flow rate comprises:
using the determined parameter of the gas present in the composite outflow determined at the first flow rate and determined at the second flow rate.

44. An apparatus according to any one of claims 39 to 43 wherein the parameter comprises a fraction of the gas component in the exhaled gas flow.

45. An apparatus according to any one of claims 39 to 44 wherein the gas is:
CO₂,
O₂,
Nitrogen,
Helium, and/or
Anaesthetic agent such as sevoflurane
and/or
the sensor is configured to sense one or more of the following in the composite gas outflow:
CO₂,
O₂,
Nitrogen,
Helium, and/or
anaesthetic agent such as sevoflurane.

46. An apparatus according to any one of claims 39 to 45 wherein the parameter of gas present in an exhaled gas flow is gas fraction and determining the fraction of the gas (F_{E}) present in an exhaled gas flow using the determined parameter of gas present in the composite gas outflow and the time-varying flow rate comprises determining the gas fraction as a function of:
Qₒ(t),Qₒ(t+Δt),Fₘ(t+Δt),Fₘ(t)
where
F_{E}(t) is the gas component concentration in the exhaled patient gas flow (volume fraction of expired gas)
Fₘ(t), fraction of the gas component measured in the patient composite gas outflow at time t
Qₒ(t), flow rate of the apparatus gas flow provided to the patient (apparatus gas flow rate), at time t
Fₘ(t+Δt), fraction of the gas component measured in the patient composite gas outflow (this is the measured CO₂/O₂ fraction parameter of the composite gas outflow) at time t+Δt
Qₒ(t+Δt), flow rate of the apparatus gas flow provided to the patient (apparatus gas flow flow rate) at time t+Δt

47. An apparatus according to any one of claims 39 to 46 wherein the parameter of the gas present in the exhaled gas flow is gas fraction and the gas is preferably CO₂ and determining the gas fraction (F_{E}) in the exhaled gas flow using the determined parameter of gas present in the composite gas outflow and the time-varying flow rate comprises using:
$${F}_{E} ( t ) ∼ {F}_{m} ( t + Δ t ) {F}_{m} ( t ) \cdot \frac{{Q}_{o} ( t + Δ t ) - {Q}_{o} ( t )}{{Q}_{o} ( t + Δ t ) {F}_{m} ( t + Δ t ) - {Q}_{o} ( t ) {F}_{m} ( t )} \qquad ( 5 )$$
Where
F_{E}(t) is the CO₂ or O₂ or other gas in the exhaled patient gas flow (volume fraction of expired gas) at time t
Fₘ(t), fraction of CO₂ measured in the composite gas outflow at time t
Qₒ(t), flow rate of the apparatus gas flow provided to the patient (apparatus gas flow flow rate) at time t
Fₘ(t+Δt), fraction of CO₂ measured in the composite gas outflow at time t+Δt
Qₒ(t+Δt), flow rate of the apparatus gas flow provided to the patient (apparatus gas flow flow rate) at time t+Δt

48. An apparatus according to any one of claims 39 to 47 wherein the sensor is positioned to measure the parameter of the gas present in the composite gas outflow from the patient at or near the mouth and/or nose of the patient.

49. An apparatus for providing an apparatus gas flow and determining a parameter of gas present in an exhaled patient gas flow, comprising:
a flow source,
a sensor for sensing a composite gas outflow, and
a controller,
wherein the apparatus is configured to:
provide an apparatus gas flow with a time-varying gas proportion (for example gas fraction),
determine a parameter of a gas present in a composite gas outflow from the patient, the composite gas outflow comprising:
leak gas flow from apparatus gas flow, and
exhaled gas flow from a patient with the gas,
and
determine the parameter of the gas present in the exhaled gas flow using the determined parameter of gas present in the composite gas outflow and the time-varying gas proportion (for example gas fraction).

50. An apparatus according to claim 49 wherein the apparatus gas flow with the time-varying gas fraction comprises at least a first gas fraction at a first time and a second gas fraction at a second time, and
determining the parameter of the gas present in the exhaled gas flow using the determined parameter of the gas present in the composite gas outflow and the time-varying gas fraction comprises:
using the determined parameter of the gas present in the composite outflow determined at the first gas fraction and determined at the second gas fraction.

51. An apparatus according to any one of claims 49 to 50 wherein the parameter comprises a fraction of the gas component in the exhaled gas flow.

52. An apparatus according to any one of claims 49 to 51 wherein the gas is:
CO₂,
O₂ ,
Nitrogen,
Helium, and/or
Anaesthetic agent such as sevoflurane.

53. An apparatus according to any one of claims 49 to 51 wherein the parameter of gas present in an exhaled gas flow is gas fraction and determining the fraction of the gas (F_{E}) present in an exhaled gas flow using the determined parameter of gas present in the composite gas outflow and the time-varying gas fraction comprises determining the gas fraction as a function of:
Fₒ(t),Fₒ(t+Δt),Fₘ(t+Δt),Fₘ(t)
Where
F_{E}(t) is the gas component concentration in the exhaled patient gas flow (volume fraction of expired gas) at time t
Fₘ(t), fraction of the gas component measured in the patient composite gas outflow at time t
Fₒ(t), gas fraction of the apparatus gas flow provided to the patient (apparatus gas flow gas fraction), at time t
Fₘ(t+Δt), fraction of the gas component measured in the patient composite gas outflow (this is the measured CO₂/O₂ fraction parameter of the composite gas outflow) at time t+Δt
Fₒ(t+Δt), gas fraction of the apparatus gas flow provided to the patient (apparatus gas flow gas fraction) at time t+Δt.

54. An apparatus according to any one of claims 49 to 53 wherein the parameter of the gas present in the exhaled gas flow is gas fraction and the gas is preferably CO₂, O₂ , Nitrogen, Helium and/or Anaesthetic agent such as sevoflurane and determining the gas fraction (F_{E}) in the exhaled gas flow using the determined parameter of gas present in the composite gas outflow and the time-varying gas fraction comprises using:
$${F}_{E} ( t ) = \frac{{F}_{m} ( t + Δ t ) ( {F}_{o} ( t ) - {F}_{m} ( t ) ) - {F}_{m} ( t ) ( {F}_{o} ( t + Δ t ) - {F}_{m} ( t + Δ t ) )}{( {F}_{o} ( t ) - {F}_{m} ( t ) ) - ( ( {F}_{o} ( t + Δ t ) - {F}_{m} ( t + Δ t ) )} \qquad ( 6 )$$
Where
F_{E}(t) is the CO₂ or O₂ or other gas in the exhaled patient gas flow (volume fraction of expired gas) at time t
Fₘ(t), fraction of CO₂ measured in the composite gas outflow at time t
Fₒ(t), gas fraction of the apparatus gas flow provided to the patient (apparatus gas flow gas fraction) at time t
Fₘ(t+Δt), fraction of CO₂ measured in the composite gas outflow at time t+Δt
Fₒ(t+Δt), gas fraction of the apparatus gas flow provided to the patient (apparatus gas flow gas fraction) at time t+Δt
Qₒ is the flow rate of the apparatus gas flow
F_{E} is the fraction of the gas in the exhaled gas flow

55. An apparatus according to any one of claims 49 to 54 wherein the sensor is positioned to measure the parameter of the gas present in the composite gas outflow from the patient at or near the mouth and/or nose of the patient.

56. A method of determining a O₂ and/or CO₂ fraction present in an exhaled gas flow comprising:
providing a humidified high flow apparatus gas flow with a time-varying flow rate to a patient via a non-sealing nasal cannula,
measuring a fraction of O₂ and/or CO₂ present in a composite gas outflow from the patient,
and
determining the fraction O₂ and/or CO₂ present in the exhaled gas flow using the measured fraction of O₂ or fraction of CO₂ present in the composite gas outflow and the time-varying flow rate.

57. An apparatus according to claim 2 wherein the sensor is senses composite gas flow by sensing gas flow the patient's:
mouth and nose,
mouth, or
nose.
